SEQUENCE LISTING (55 sequences)

SEQ ID NO: 1 - DNA, 10 bp, Artificial Sequence
  stress inducible element
gtgagagagc

SEQ ID NO: 2 - DNA, 10 bp, Artificial Sequence
  stress inducible element
gdgagagagn

SEQ ID NO: 3 - DNA, 20 bp, Artificial Sequence
  repitition motif comprising stress inducible element
tattttgtgagagagctcca

SEQ ID NO: 4 - DNA, 158 bp, Artificial Sequence
  Artificial Promoter
tattttgtgagagagctccatattttgtgagagagctccatattttgtgagagagctccatattttgtgagagagctccatctagagtcgaccgcaagacccttcctctatataaggaagttcatttcatttggagaggacacgctcgagtggccacc

SEQ ID NO: 5 - DNA, 13662 bp, Artificial Sequence
  Expression vector
ttgtacaaagtggtgatatcacaagcccgggcggtcttctagggataacagggtaattatatccctctagatcacaagcccgggcggtcttctacgatgattgagtaataatgtgtcacgcatcaccatgggtggcagtgtcagtgtgagcaatgacctgaatgaacaattgaaatgaaaagaaaaaaagtactccatctgttccaaattaaaattggttttaaccttttaataggtttatacaataattgatatatgttttctgtatatgtctaatttgttatcatccgggcggtcttctagggataacagggtaattatatccctctagacaacacacaacaaataagagaaaaaacaaataatattaatttgagaatgaacaaaaggaccatatcattcattaactcttctccatccacttccatttcacagttcgatagcgaaaaccgaataaaaaacacagtaaattacaagcacaacaaatggtacaagaaaaacagttttcccaatgccataatactcgactcgagttcctgcaggtaccaaaagcttagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtatgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgcttagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattcaagagctcaaggatcctaactataacggtcctaaggtagcgaaggcgcgccgaattcgaggggatcgagcccctgctgagcctcgacatgttgtcgcaaaattcgccctggacccgcccaacgatttgtcgtcactgtcaaggtttgacctgcacttcatttggggcccacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgagcgttattagttcgccgctcggtgtgtcgtagatactagcccctggggcacttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcagggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccacatgatcaaacgttttgaggacgcgagaggattcgattcgacgacgagagcctcgcgagattggggagaaatttttcgggggtggagctgatgcgaggagaggagatgagggggctggtatttatggcggttgggtggtgggaggagtcccgtgccgtgacgtctccgtctgcttggagaatccgccacgctgaaaccaccgcggtttccgggaagacgaggcgggcgagcgagcggttgggaaatttcgagaagatgccgtttgtctccgtttggtacacgtctcgttgatttttttttagtgaattacgctttggaccacattttattatctaagggtgtgtttggttgtaagccacactttgccacagtttgccacgcctaaggttaggcaaatttgacaggtgtttggttgtagccacagttgtggcaagatttccctctaacaaattaagtcccacgtgtcaatggctcaaaaaagtgtggcaagattcccttaggcttagtaagttgtggctaacaatttgatcacctcaccttagacaaggtgtggcaacttttgttggcaagtaatggtaaagtatggctgggaaccaaacagcccctaagttttactttggactacctttaaacatatcttttcactttgaactagataaatttgctattgttgcgatttggattttttttttctcgtgcaatcaacgaccttaaacacatcagctctagtatacggccgatctcctctatatatggttcatatgtttgccgaaagggaagttagacatgacgaaaagttgttcatggtagtccaaaccacaacccggcccaatttgaaaagataggtttaagggtggtccaaattgaaacttgggtaataaaaggtggatcaaagtgcaatttacttttttttactgtaatttcttctggctggtttgttggtcgccgttaggaccgggtgacgccgtcaaccccgcgcctccgtattcgctgacgtggggtggcgcgctggcttccgccttgacccgaatttgttttccttccgttaaaaaaatggttttccttttcttaaaaaggaaatagtttgttttttaagtctgtgtattaggattattacacttgaattttggtatatgtgtaggataatttactgcatgtttataatagagttgtactatagatgaaataacccaatttttggtataattcgtgtttggttggaggtcaaaataacaggttattttgtgaagaaaaaactccgtagtatagtaccatatccatcatgaatacacatactgcctagacgagtgattaggatgaatccatgttatattcctcaaaataatataaaccacttgatcttatgatcttatccaatctgttcatataaactggagatataagatggtgcatttcccttttgatttcttttgttgacggccatgagataggttgcatccactgcatttatattttggaccaatacaatgcacctattgatacatggggacagctcaactaaccatgatgcaaaatgctggttggtgaccagttcttggcattatgataatgataggattaaaaaaaacagtgcaatgtctcggaaagaaaccatgacaaagggtacatgttgcattccagtttctaatgataaaattatgtgccagcaattcaaaaatcatgcgtgttccctacgcaccattctttgcaataaacaagtgcatgcacaatatgattgtgctaaggttcaagaacttgttgcagtggctaagcttggcgcgcctcgcgaccacctttaattaagtgaagagcaggagcttgcatgcctgcaggctctagaggatcccccctcagaagaccagagggctattgagacttttcaacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacaggcttcttgagatccttcaacaattaccaacaacaacaaacaacaaacaacattacaattactatttacaattacagtcgactctagaggatccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaaagcggccgcccggctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgatgataagctgtcaaacatgagaattcctttcgtcgacccacgtgttgctgaggtatttaaattattttgtgagagagctccatattttgtgagagagctccatattttgtgagagagctccatattttgtgagagagctccatctagagtcgaccgcaagacccttcctctatataaggaagttcatttcatttggagaggacacgctcgagtggccaccatttaaatcaactagggatatcacaagtttgtacaaaaaagcaggcttcacaatgttacgtcctgtagaaaccccaacccgtgaaatcaaaaaactcgacggcctgtgggcattcagtctggatcgcgaaaactgtggaattggtcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaaacccagctttc

SEQ ID NO: 6 - DNA, 26 bp, Artificial Sequence
  stress inducible element
aatcgggacccacgtcgcactctacc

SEQ ID NO: 7 - DNA, 11 bp, Artificial Sequence
  stress inducible element
sncacgtcrnn

SEQ ID NO: 8 - DNA, 30 bp, Artificial Sequence
  repitition motif comprising stress inducible element
gcaatcgggacccacgtcgcactctacccc

SEQ ID NO: 9 - DNA, 198 bp, Artificial Sequence
  Artificial Promoter
gcaatcgggacccacgtcgcactctaccccgcaatcgggacccacgtcgcactctaccccgcaatcgggacccacgtcgcactctaccccgcaatcgggacccacgtcgcactctacccctctagagtcgaccgcaagacccttcctctatataaggaagttcatttcatttggagaggacacgctcgagtggccacc

SEQ ID NO: 10 - DNA, 13702 bp, Artificial Sequence
  Expression vector
ttgtacaaagtggtgatatcacaagcccgggcggtcttctagggataacagggtaattatatccctctagatcacaagcccgggcggtcttctacgatgattgagtaataatgtgtcacgcatcaccatgggtggcagtgtcagtgtgagcaatgacctgaatgaacaattgaaatgaaaagaaaaaaagtactccatctgttccaaattaaaattggttttaaccttttaataggtttatacaataattgatatatgttttctgtatatgtctaatttgttatcatccgggcggtcttctagggataacagggtaattatatccctctagacaacacacaacaaataagagaaaaaacaaataatattaatttgagaatgaacaaaaggaccatatcattcattaactcttctccatccacttccatttcacagttcgatagcgaaaaccgaataaaaaacacagtaaattacaagcacaacaaatggtacaagaaaaacagttttcccaatgccataatactcgactcgagttcctgcaggtaccaaaagcttagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtatgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgcttagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattcaagagctcaaggatcctaactataacggtcctaaggtagcgaaggcgcgccgaattcgaggggatcgagcccctgctgagcctcgacatgttgtcgcaaaattcgccctggacccgcccaacgatttgtcgtcactgtcaaggtttgacctgcacttcatttggggcccacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgagcgttattagttcgccgctcggtgtgtcgtagatactagcccctggggcacttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcagggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccacatgatcaaacgttttgaggacgcgagaggattcgattcgacgacgagagcctcgcgagattggggagaaatttttcgggggtggagctgatgcgaggagaggagatgagggggctggtatttatggcggttgggtggtgggaggagtcccgtgccgtgacgtctccgtctgcttggagaatccgccacgctgaaaccaccgcggtttccgggaagacgaggcgggcgagcgagcggttgggaaatttcgagaagatgccgtttgtctccgtttggtacacgtctcgttgatttttttttagtgaattacgctttggaccacattttattatctaagggtgtgtttggttgtaagccacactttgccacagtttgccacgcctaaggttaggcaaatttgacaggtgtttggttgtagccacagttgtggcaagatttccctctaacaaattaagtcccacgtgtcaatggctcaaaaaagtgtggcaagattcccttaggcttagtaagttgtggctaacaatttgatcacctcaccttagacaaggtgtggcaacttttgttggcaagtaatggtaaagtatggctgggaaccaaacagcccctaagttttactttggactacctttaaacatatcttttcactttgaactagataaatttgctattgttgcgatttggattttttttttctcgtgcaatcaacgaccttaaacacatcagctctagtatacggccgatctcctctatatatggttcatatgtttgccgaaagggaagttagacatgacgaaaagttgttcatggtagtccaaaccacaacccggcccaatttgaaaagataggtttaagggtggtccaaattgaaacttgggtaataaaaggtggatcaaagtgcaatttacttttttttactgtaatttcttctggctggtttgttggtcgccgttaggaccgggtgacgccgtcaaccccgcgcctccgtattcgctgacgtggggtggcgcgctggcttccgccttgacccgaatttgttttccttccgttaaaaaaatggttttccttttcttaaaaaggaaatagtttgttttttaagtctgtgtattaggattattacacttgaattttggtatatgtgtaggataatttactgcatgtttataatagagttgtactatagatgaaataacccaatttttggtataattcgtgtttggttggaggtcaaaataacaggttattttgtgaagaaaaaactccgtagtatagtaccatatccatcatgaatacacatactgcctagacgagtgattaggatgaatccatgttatattcctcaaaataatataaaccacttgatcttatgatcttatccaatctgttcatataaactggagatataagatggtgcatttcccttttgatttcttttgttgacggccatgagataggttgcatccactgcatttatattttggaccaatacaatgcacctattgatacatggggacagctcaactaaccatgatgcaaaatgctggttggtgaccagttcttggcattatgataatgataggattaaaaaaaacagtgcaatgtctcggaaagaaaccatgacaaagggtacatgttgcattccagtttctaatgataaaattatgtgccagcaattcaaaaatcatgcgtgttccctacgcaccattctttgcaataaacaagtgcatgcacaatatgattgtgctaaggttcaagaacttgttgcagtggctaagcttggcgcgcctcgcgaccacctttaattaagtgaagagcaggagcttgcatgcctgcaggctctagaggatcccccctcagaagaccagagggctattgagacttttcaacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacaggcttcttgagatccttcaacaattaccaacaacaacaaacaacaaacaacattacaattactatttacaattacagtcgactctagaggatccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaaagcggccgcccggctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgatgataagctgtcaaacatgagaattcctttcgtcgacccacgtgttgctgaggtatttaaatgcaatcgggacccacgtcgcactctaccccgcaatcgggacccacgtcgcactctaccccgcaatcgggacccacgtcgcactctaccccgcaatcgggacccacgtcgcactctacccctctagagtcgaccgcaagacccttcctctatataaggaagttcatttcatttggagaggacacgctcgagtggccaccatttaaatcaactagggatatcacaagtttgtacaaaaaagcaggcttcacaatgttacgtcctgtagaaaccccaacccgtgaaatcaaaaaactcgacggcctgtgggcattcagtctggatcgcgaaaactgtggaattggtcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaaacccagctttc

SEQ ID NO: 11 - DNA, 18 bp, Artificial Sequence
  stress inducible element
tttgttggagtcgcaatc

SEQ ID NO: 12 - DNA, 18 bp, Artificial Sequence
  stress inducible element
tttgttggwbnyaywytm

SEQ ID NO: 13 - DNA, 20 bp, Artificial Sequence
  repitition motif comprising stress inducible element
gtttgttggagtcgcaatca

SEQ ID NO: 14 - DNA, 158 bp, Artificial Sequence
  Artificial Promoter
gtttgttggagtcgcaatcagtttgttggagtcgcaatcagtttgttggagtcgcaatcagtttgttggagtcgcaatcatctagagtcgaccgcaagacccttcctctatataaggaagttcatttcatttggagaggacacgctcgagtggccacc

SEQ ID NO: 15 - DNA, 13662 bp, Artificial Sequence
  Expression vector
ttgtacaaagtggtgatatcacaagcccgggcggtcttctagggataacagggtaattatatccctctagatcacaagcccgggcggtcttctacgatgattgagtaataatgtgtcacgcatcaccatgggtggcagtgtcagtgtgagcaatgacctgaatgaacaattgaaatgaaaagaaaaaaagtactccatctgttccaaattaaaattggttttaaccttttaataggtttatacaataattgatatatgttttctgtatatgtctaatttgttatcatccgggcggtcttctagggataacagggtaattatatccctctagacaacacacaacaaataagagaaaaaacaaataatattaatttgagaatgaacaaaaggaccatatcattcattaactcttctccatccacttccatttcacagttcgatagcgaaaaccgaataaaaaacacagtaaattacaagcacaacaaatggtacaagaaaaacagttttcccaatgccataatactcgactcgagttcctgcaggtaccaaaagcttagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtatgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgcttagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattcaagagctcaaggatcctaactataacggtcctaaggtagcgaaggcgcgccgaattcgaggggatcgagcccctgctgagcctcgacatgttgtcgcaaaattcgccctggacccgcccaacgatttgtcgtcactgtcaaggtttgacctgcacttcatttggggcccacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgagcgttattagttcgccgctcggtgtgtcgtagatactagcccctggggcacttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcagggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccacatgatcaaacgttttgaggacgcgagaggattcgattcgacgacgagagcctcgcgagattggggagaaatttttcgggggtggagctgatgcgaggagaggagatgagggggctggtatttatggcggttgggtggtgggaggagtcccgtgccgtgacgtctccgtctgcttggagaatccgccacgctgaaaccaccgcggtttccgggaagacgaggcgggcgagcgagcggttgggaaatttcgagaagatgccgtttgtctccgtttggtacacgtctcgttgatttttttttagtgaattacgctttggaccacattttattatctaagggtgtgtttggttgtaagccacactttgccacagtttgccacgcctaaggttaggcaaatttgacaggtgtttggttgtagccacagttgtggcaagatttccctctaacaaattaagtcccacgtgtcaatggctcaaaaaagtgtggcaagattcccttaggcttagtaagttgtggctaacaatttgatcacctcaccttagacaaggtgtggcaacttttgttggcaagtaatggtaaagtatggctgggaaccaaacagcccctaagttttactttggactacctttaaacatatcttttcactttgaactagataaatttgctattgttgcgatttggattttttttttctcgtgcaatcaacgaccttaaacacatcagctctagtatacggccgatctcctctatatatggttcatatgtttgccgaaagggaagttagacatgacgaaaagttgttcatggtagtccaaaccacaacccggcccaatttgaaaagataggtttaagggtggtccaaattgaaacttgggtaataaaaggtggatcaaagtgcaatttacttttttttactgtaatttcttctggctggtttgttggtcgccgttaggaccgggtgacgccgtcaaccccgcgcctccgtattcgctgacgtggggtggcgcgctggcttccgccttgacccgaatttgttttccttccgttaaaaaaatggttttccttttcttaaaaaggaaatagtttgttttttaagtctgtgtattaggattattacacttgaattttggtatatgtgtaggataatttactgcatgtttataatagagttgtactatagatgaaataacccaatttttggtataattcgtgtttggttggaggtcaaaataacaggttattttgtgaagaaaaaactccgtagtatagtaccatatccatcatgaatacacatactgcctagacgagtgattaggatgaatccatgttatattcctcaaaataatataaaccacttgatcttatgatcttatccaatctgttcatataaactggagatataagatggtgcatttcccttttgatttcttttgttgacggccatgagataggttgcatccactgcatttatattttggaccaatacaatgcacctattgatacatggggacagctcaactaaccatgatgcaaaatgctggttggtgaccagttcttggcattatgataatgataggattaaaaaaaacagtgcaatgtctcggaaagaaaccatgacaaagggtacatgttgcattccagtttctaatgataaaattatgtgccagcaattcaaaaatcatgcgtgttccctacgcaccattctttgcaataaacaagtgcatgcacaatatgattgtgctaaggttcaagaacttgttgcagtggctaagcttggcgcgcctcgcgaccacctttaattaagtgaagagcaggagcttgcatgcctgcaggctctagaggatcccccctcagaagaccagagggctattgagacttttcaacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacaggcttcttgagatccttcaacaattaccaacaacaacaaacaacaaacaacattacaattactatttacaattacagtcgactctagaggatccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaaagcggccgcccggctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgatgataagctgtcaaacatgagaattcctttcgtcgacccacgtgttgctgaggtatttaaatgtttgttggagtcgcaatcagtttgttggagtcgcaatcagtttgttggagtcgcaatcagtttgttggagtcgcaatcatctagagtcgaccgcaagacccttcctctatataaggaagttcatttcatttggagaggacacgctcgagtggccaccatttaaatcaactagggatatcacaagtttgtacaaaaaagcaggcttcacaatgttacgtcctgtagaaaccccaacccgtgaaatcaaaaaactcgacggcctgtgggcattcagtctggatcgcgaaaactgtggaattggtcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaaacccagctttc

SEQ ID NO: 16 - DNA, 22 bp, Artificial Sequence
  stress inducible element
gcctctctccacctcgcccacg

SEQ ID NO: 17 - DNA, 15 bp, Artificial Sequence
  stress inducible element
ycyhcnctccwccnc

SEQ ID NO: 18 - DNA, 30 bp, Artificial Sequence
  repitition motif comprising stress inducible element
ccacgcctctctccacctcgcccacgcaca

SEQ ID NO: 19 - DNA, 198 bp, Artificial Sequence
  Artificial Promoter
ccacgcctctctccacctcgcccacgcacaccacgcctctctccacctcgcccacgcacaccacgcctctctccacctcgcccacgcacaccacgcctctctccacctcgcccacgcacatctagagtcgaccgcaagacccttcctctatataaggaagttcatttcatttggagaggacacgctcgagtggccacc

SEQ ID NO: 20 - DNA, 13702 bp, Artificial Sequence
  Expression vector
ttgtacaaagtggtgatatcacaagcccgggcggtcttctagggataacagggtaattatatccctctagatcacaagcccgggcggtcttctacgatgattgagtaataatgtgtcacgcatcaccatgggtggcagtgtcagtgtgagcaatgacctgaatgaacaattgaaatgaaaagaaaaaaagtactccatctgttccaaattaaaattggttttaaccttttaataggtttatacaataattgatatatgttttctgtatatgtctaatttgttatcatccgggcggtcttctagggataacagggtaattatatccctctagacaacacacaacaaataagagaaaaaacaaataatattaatttgagaatgaacaaaaggaccatatcattcattaactcttctccatccacttccatttcacagttcgatagcgaaaaccgaataaaaaacacagtaaattacaagcacaacaaatggtacaagaaaaacagttttcccaatgccataatactcgactcgagttcctgcaggtaccaaaagcttagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtatgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgcttagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattcaagagctcaaggatcctaactataacggtcctaaggtagcgaaggcgcgccgaattcgaggggatcgagcccctgctgagcctcgacatgttgtcgcaaaattcgccctggacccgcccaacgatttgtcgtcactgtcaaggtttgacctgcacttcatttggggcccacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgagcgttattagttcgccgctcggtgtgtcgtagatactagcccctggggcacttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcagggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccacatgatcaaacgttttgaggacgcgagaggattcgattcgacgacgagagcctcgcgagattggggagaaatttttcgggggtggagctgatgcgaggagaggagatgagggggctggtatttatggcggttgggtggtgggaggagtcccgtgccgtgacgtctccgtctgcttggagaatccgccacgctgaaaccaccgcggtttccgggaagacgaggcgggcgagcgagcggttgggaaatttcgagaagatgccgtttgtctccgtttggtacacgtctcgttgatttttttttagtgaattacgctttggaccacattttattatctaagggtgtgtttggttgtaagccacactttgccacagtttgccacgcctaaggttaggcaaatttgacaggtgtttggttgtagccacagttgtggcaagatttccctctaacaaattaagtcccacgtgtcaatggctcaaaaaagtgtggcaagattcccttaggcttagtaagttgtggctaacaatttgatcacctcaccttagacaaggtgtggcaacttttgttggcaagtaatggtaaagtatggctgggaaccaaacagcccctaagttttactttggactacctttaaacatatcttttcactttgaactagataaatttgctattgttgcgatttggattttttttttctcgtgcaatcaacgaccttaaacacatcagctctagtatacggccgatctcctctatatatggttcatatgtttgccgaaagggaagttagacatgacgaaaagttgttcatggtagtccaaaccacaacccggcccaatttgaaaagataggtttaagggtggtccaaattgaaacttgggtaataaaaggtggatcaaagtgcaatttacttttttttactgtaatttcttctggctggtttgttggtcgccgttaggaccgggtgacgccgtcaaccccgcgcctccgtattcgctgacgtggggtggcgcgctggcttccgccttgacccgaatttgttttccttccgttaaaaaaatggttttccttttcttaaaaaggaaatagtttgttttttaagtctgtgtattaggattattacacttgaattttggtatatgtgtaggataatttactgcatgtttataatagagttgtactatagatgaaataacccaatttttggtataattcgtgtttggttggaggtcaaaataacaggttattttgtgaagaaaaaactccgtagtatagtaccatatccatcatgaatacacatactgcctagacgagtgattaggatgaatccatgttatattcctcaaaataatataaaccacttgatcttatgatcttatccaatctgttcatataaactggagatataagatggtgcatttcccttttgatttcttttgttgacggccatgagataggttgcatccactgcatttatattttggaccaatacaatgcacctattgatacatggggacagctcaactaaccatgatgcaaaatgctggttggtgaccagttcttggcattatgataatgataggattaaaaaaaacagtgcaatgtctcggaaagaaaccatgacaaagggtacatgttgcattccagtttctaatgataaaattatgtgccagcaattcaaaaatcatgcgtgttccctacgcaccattctttgcaataaacaagtgcatgcacaatatgattgtgctaaggttcaagaacttgttgcagtggctaagcttggcgcgcctcgcgaccacctttaattaagtgaagagcaggagcttgcatgcctgcaggctctagaggatcccccctcagaagaccagagggctattgagacttttcaacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacaggcttcttgagatccttcaacaattaccaacaacaacaaacaacaaacaacattacaattactatttacaattacagtcgactctagaggatccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaaagcggccgcccggctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgatgataagctgtcaaacatgagaattcctttcgtcgacccacgtgttgctgaggtatttaaatccacgcctctctccacctcgcccacgcacaccacgcctctctccacctcgcccacgcacaccacgcctctctccacctcgcccacgcacaccacgcctctctccacctcgcccacgcacatctagagtcgaccgcaagacccttcctctatataaggaagttcatttcatttggagaggacacgctcgagtggccaccatttaaatcaactagggatatcacaagtttgtacaaaaaagcaggcttcacaatgttacgtcctgtagaaaccccaacccgtgaaatcaaaaaactcgacggcctgtgggcattcagtctggatcgcgaaaactgtggaattggtcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaaacccagctttc

SEQ ID NO: 21 - DNA, 20 bp, Artificial Sequence
  stress inducible element
caaaatgtgtgcacaacaac

SEQ ID NO: 22 - DNA, 17 bp, Artificial Sequence
  stress inducible element
aannmngngasaacaan

SEQ ID NO: 23 - DNA, 20 bp, Artificial Sequence
  repitition motif comprising stress inducible element
caaaatgtgtgcacaacaac

SEQ ID NO: 24 - DNA, 158 bp, Artificial Sequence
  Artificial Promoter
caaaatgtgtgcacaacaaccaaaatgtgtgcacaacaaccaaaatgtgtgcacaacaaccaaaatgtgtgcacaacaactctagagtcgaccgcaagacccttcctctatataaggaagttcatttcatttggagaggacacgctcgagtggccacc

SEQ ID NO: 25 - DNA, 13662 bp, Artificial Sequence
  Expression vector
ttgtacaaagtggtgatatcacaagcccgggcggtcttctagggataacagggtaattatatccctctagatcacaagcccgggcggtcttctacgatgattgagtaataatgtgtcacgcatcaccatgggtggcagtgtcagtgtgagcaatgacctgaatgaacaattgaaatgaaaagaaaaaaagtactccatctgttccaaattaaaattggttttaaccttttaataggtttatacaataattgatatatgttttctgtatatgtctaatttgttatcatccgggcggtcttctagggataacagggtaattatatccctctagacaacacacaacaaataagagaaaaaacaaataatattaatttgagaatgaacaaaaggaccatatcattcattaactcttctccatccacttccatttcacagttcgatagcgaaaaccgaataaaaaacacagtaaattacaagcacaacaaatggtacaagaaaaacagttttcccaatgccataatactcgactcgagttcctgcaggtaccaaaagcttagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtatgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgcttagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattcaagagctcaaggatcctaactataacggtcctaaggtagcgaaggcgcgccgaattcgaggggatcgagcccctgctgagcctcgacatgttgtcgcaaaattcgccctggacccgcccaacgatttgtcgtcactgtcaaggtttgacctgcacttcatttggggcccacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgagcgttattagttcgccgctcggtgtgtcgtagatactagcccctggggcacttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcagggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccacatgatcaaacgttttgaggacgcgagaggattcgattcgacgacgagagcctcgcgagattggggagaaatttttcgggggtggagctgatgcgaggagaggagatgagggggctggtatttatggcggttgggtggtgggaggagtcccgtgccgtgacgtctccgtctgcttggagaatccgccacgctgaaaccaccgcggtttccgggaagacgaggcgggcgagcgagcggttgggaaatttcgagaagatgccgtttgtctccgtttggtacacgtctcgttgatttttttttagtgaattacgctttggaccacattttattatctaagggtgtgtttggttgtaagccacactttgccacagtttgccacgcctaaggttaggcaaatttgacaggtgtttggttgtagccacagttgtggcaagatttccctctaacaaattaagtcccacgtgtcaatggctcaaaaaagtgtggcaagattcccttaggcttagtaagttgtggctaacaatttgatcacctcaccttagacaaggtgtggcaacttttgttggcaagtaatggtaaagtatggctgggaaccaaacagcccctaagttttactttggactacctttaaacatatcttttcactttgaactagataaatttgctattgttgcgatttggattttttttttctcgtgcaatcaacgaccttaaacacatcagctctagtatacggccgatctcctctatatatggttcatatgtttgccgaaagggaagttagacatgacgaaaagttgttcatggtagtccaaaccacaacccggcccaatttgaaaagataggtttaagggtggtccaaattgaaacttgggtaataaaaggtggatcaaagtgcaatttacttttttttactgtaatttcttctggctggtttgttggtcgccgttaggaccgggtgacgccgtcaaccccgcgcctccgtattcgctgacgtggggtggcgcgctggcttccgccttgacccgaatttgttttccttccgttaaaaaaatggttttccttttcttaaaaaggaaatagtttgttttttaagtctgtgtattaggattattacacttgaattttggtatatgtgtaggataatttactgcatgtttataatagagttgtactatagatgaaataacccaatttttggtataattcgtgtttggttggaggtcaaaataacaggttattttgtgaagaaaaaactccgtagtatagtaccatatccatcatgaatacacatactgcctagacgagtgattaggatgaatccatgttatattcctcaaaataatataaaccacttgatcttatgatcttatccaatctgttcatataaactggagatataagatggtgcatttcccttttgatttcttttgttgacggccatgagataggttgcatccactgcatttatattttggaccaatacaatgcacctattgatacatggggacagctcaactaaccatgatgcaaaatgctggttggtgaccagttcttggcattatgataatgataggattaaaaaaaacagtgcaatgtctcggaaagaaaccatgacaaagggtacatgttgcattccagtttctaatgataaaattatgtgccagcaattcaaaaatcatgcgtgttccctacgcaccattctttgcaataaacaagtgcatgcacaatatgattgtgctaaggttcaagaacttgttgcagtggctaagcttggcgcgcctcgcgaccacctttaattaagtgaagagcaggagcttgcatgcctgcaggctctagaggatcccccctcagaagaccagagggctattgagacttttcaacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacaggcttcttgagatccttcaacaattaccaacaacaacaaacaacaaacaacattacaattactatttacaattacagtcgactctagaggatccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaaagcggccgcccggctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgatgataagctgtcaaacatgagaattcctttcgtcgacccacgtgttgctgaggtatttaaatcaaaatgtgtgcacaacaaccaaaatgtgtgcacaacaaccaaaatgtgtgcacaacaaccaaaatgtgtgcacaacaactctagagtcgaccgcaagacccttcctctatataaggaagttcatttcatttggagaggacacgctcgagtggccaccatttaaatcaactagggatatcacaagtttgtacaaaaaagcaggcttcacaatgttacgtcctgtagaaaccccaacccgtgaaatcaaaaaactcgacggcctgtgggcattcagtctggatcgcgaaaactgtggaattggtcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaaacccagctttc

SEQ ID NO: 26 - DNA, 27 bp, Artificial Sequence
  stress inducible element
tcttgactttggaccctgccccactat

SEQ ID NO: 27 - DNA, 27 bp, Artificial Sequence
  stress inducible element
dywngdnkttggtccnakynnmannch

SEQ ID NO: 28 - DNA, 30 bp, Artificial Sequence
  repitition motif comprising stress inducible element
tttcttgactttggaccctgccccactatc

SEQ ID NO: 29 - DNA, 198 bp, Artificial Sequence
  Artificial Promoter
tttcttgactttggaccctgccccactatctttcttgactttggaccctgccccactatctttcttgactttggaccctgccccactatctttcttgactttggaccctgccccactatctctagagtcgaccgcaagacccttcctctatataaggaagttcatttcatttggagaggacacgctcgagtggccacc

SEQ ID NO: 30 - DNA, 13702 bp, Artificial Sequence
  Expression vector
ttgtacaaagtggtgatatcacaagcccgggcggtcttctagggataacagggtaattatatccctctagatcacaagcccgggcggtcttctacgatgattgagtaataatgtgtcacgcatcaccatgggtggcagtgtcagtgtgagcaatgacctgaatgaacaattgaaatgaaaagaaaaaaagtactccatctgttccaaattaaaattggttttaaccttttaataggtttatacaataattgatatatgttttctgtatatgtctaatttgttatcatccgggcggtcttctagggataacagggtaattatatccctctagacaacacacaacaaataagagaaaaaacaaataatattaatttgagaatgaacaaaaggaccatatcattcattaactcttctccatccacttccatttcacagttcgatagcgaaaaccgaataaaaaacacagtaaattacaagcacaacaaatggtacaagaaaaacagttttcccaatgccataatactcgactcgagttcctgcaggtaccaaaagcttagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtatgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgcttagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattcaagagctcaaggatcctaactataacggtcctaaggtagcgaaggcgcgccgaattcgaggggatcgagcccctgctgagcctcgacatgttgtcgcaaaattcgccctggacccgcccaacgatttgtcgtcactgtcaaggtttgacctgcacttcatttggggcccacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgagcgttattagttcgccgctcggtgtgtcgtagatactagcccctggggcacttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcagggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccacatgatcaaacgttttgaggacgcgagaggattcgattcgacgacgagagcctcgcgagattggggagaaatttttcgggggtggagctgatgcgaggagaggagatgagggggctggtatttatggcggttgggtggtgggaggagtcccgtgccgtgacgtctccgtctgcttggagaatccgccacgctgaaaccaccgcggtttccgggaagacgaggcgggcgagcgagcggttgggaaatttcgagaagatgccgtttgtctccgtttggtacacgtctcgttgatttttttttagtgaattacgctttggaccacattttattatctaagggtgtgtttggttgtaagccacactttgccacagtttgccacgcctaaggttaggcaaatttgacaggtgtttggttgtagccacagttgtggcaagatttccctctaacaaattaagtcccacgtgtcaatggctcaaaaaagtgtggcaagattcccttaggcttagtaagttgtggctaacaatttgatcacctcaccttagacaaggtgtggcaacttttgttggcaagtaatggtaaagtatggctgggaaccaaacagcccctaagttttactttggactacctttaaacatatcttttcactttgaactagataaatttgctattgttgcgatttggattttttttttctcgtgcaatcaacgaccttaaacacatcagctctagtatacggccgatctcctctatatatggttcatatgtttgccgaaagggaagttagacatgacgaaaagttgttcatggtagtccaaaccacaacccggcccaatttgaaaagataggtttaagggtggtccaaattgaaacttgggtaataaaaggtggatcaaagtgcaatttacttttttttactgtaatttcttctggctggtttgttggtcgccgttaggaccgggtgacgccgtcaaccccgcgcctccgtattcgctgacgtggggtggcgcgctggcttccgccttgacccgaatttgttttccttccgttaaaaaaatggttttccttttcttaaaaaggaaatagtttgttttttaagtctgtgtattaggattattacacttgaattttggtatatgtgtaggataatttactgcatgtttataatagagttgtactatagatgaaataacccaatttttggtataattcgtgtttggttggaggtcaaaataacaggttattttgtgaagaaaaaactccgtagtatagtaccatatccatcatgaatacacatactgcctagacgagtgattaggatgaatccatgttatattcctcaaaataatataaaccacttgatcttatgatcttatccaatctgttcatataaactggagatataagatggtgcatttcccttttgatttcttttgttgacggccatgagataggttgcatccactgcatttatattttggaccaatacaatgcacctattgatacatggggacagctcaactaaccatgatgcaaaatgctggttggtgaccagttcttggcattatgataatgataggattaaaaaaaacagtgcaatgtctcggaaagaaaccatgacaaagggtacatgttgcattccagtttctaatgataaaattatgtgccagcaattcaaaaatcatgcgtgttccctacgcaccattctttgcaataaacaagtgcatgcacaatatgattgtgctaaggttcaagaacttgttgcagtggctaagcttggcgcgcctcgcgaccacctttaattaagtgaagagcaggagcttgcatgcctgcaggctctagaggatcccccctcagaagaccagagggctattgagacttttcaacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacaggcttcttgagatccttcaacaattaccaacaacaacaaacaacaaacaacattacaattactatttacaattacagtcgactctagaggatccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaaagcggccgcccggctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgatgataagctgtcaaacatgagaattcctttcgtcgacccacgtgttgctgaggtatttaaattttcttgactttggaccctgccccactatctttcttgactttggaccctgccccactatctttcttgactttggaccctgccccactatctttcttgactttggaccctgccccactatctctagagtcgaccgcaagacccttcctctatataaggaagttcatttcatttggagaggacacgctcgagtggccaccatttaaatcaactagggatatcacaagtttgtacaaaaaagcaggcttcacaatgttacgtcctgtagaaaccccaacccgtgaaatcaaaaaactcgacggcctgtgggcattcagtctggatcgcgaaaactgtggaattggtcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaaacccagctttc

SEQ ID NO: 31 - DNA, 12 bp, Artificial Sequence
  stress inducible element
tcttcctcttcc

SEQ ID NO: 32 - DNA, 10 bp, Artificial Sequence
  stress inducible element
ncttcctynn

SEQ ID NO: 33 - DNA, 20 bp, Artificial Sequence
  repitition motif comprising stress inducible element
cgcctcttcctcttccctca

SEQ ID NO: 34 - DNA, 158 bp, Artificial Sequence
  Artificial Promoter
cgcctcttcctcttccctcacgcctcttcctcttccctcacgcctcttcctcttccctcacgcctcttcctcttccctcatctagagtcgaccgcaagacccttcctctatataaggaagttcatttcatttggagaggacacgctcgagtggccacc

SEQ ID NO: 35 - DNA, 13662 bp, Artificial Sequence
  Expression vector
ttgtacaaagtggtgatatcacaagcccgggcggtcttctagggataacagggtaattatatccctctagatcacaagcccgggcggtcttctacgatgattgagtaataatgtgtcacgcatcaccatgggtggcagtgtcagtgtgagcaatgacctgaatgaacaattgaaatgaaaagaaaaaaagtactccatctgttccaaattaaaattggttttaaccttttaataggtttatacaataattgatatatgttttctgtatatgtctaatttgttatcatccgggcggtcttctagggataacagggtaattatatccctctagacaacacacaacaaataagagaaaaaacaaataatattaatttgagaatgaacaaaaggaccatatcattcattaactcttctccatccacttccatttcacagttcgatagcgaaaaccgaataaaaaacacagtaaattacaagcacaacaaatggtacaagaaaaacagttttcccaatgccataatactcgactcgagttcctgcaggtaccaaaagcttagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtatgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgcttagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattcaagagctcaaggatcctaactataacggtcctaaggtagcgaaggcgcgccgaattcgaggggatcgagcccctgctgagcctcgacatgttgtcgcaaaattcgccctggacccgcccaacgatttgtcgtcactgtcaaggtttgacctgcacttcatttggggcccacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgagcgttattagttcgccgctcggtgtgtcgtagatactagcccctggggcacttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcagggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccacatgatcaaacgttttgaggacgcgagaggattcgattcgacgacgagagcctcgcgagattggggagaaatttttcgggggtggagctgatgcgaggagaggagatgagggggctggtatttatggcggttgggtggtgggaggagtcccgtgccgtgacgtctccgtctgcttggagaatccgccacgctgaaaccaccgcggtttccgggaagacgaggcgggcgagcgagcggttgggaaatttcgagaagatgccgtttgtctccgtttggtacacgtctcgttgatttttttttagtgaattacgctttggaccacattttattatctaagggtgtgtttggttgtaagccacactttgccacagtttgccacgcctaaggttaggcaaatttgacaggtgtttggttgtagccacagttgtggcaagatttccctctaacaaattaagtcccacgtgtcaatggctcaaaaaagtgtggcaagattcccttaggcttagtaagttgtggctaacaatttgatcacctcaccttagacaaggtgtggcaacttttgttggcaagtaatggtaaagtatggctgggaaccaaacagcccctaagttttactttggactacctttaaacatatcttttcactttgaactagataaatttgctattgttgcgatttggattttttttttctcgtgcaatcaacgaccttaaacacatcagctctagtatacggccgatctcctctatatatggttcatatgtttgccgaaagggaagttagacatgacgaaaagttgttcatggtagtccaaaccacaacccggcccaatttgaaaagataggtttaagggtggtccaaattgaaacttgggtaataaaaggtggatcaaagtgcaatttacttttttttactgtaatttcttctggctggtttgttggtcgccgttaggaccgggtgacgccgtcaaccccgcgcctccgtattcgctgacgtggggtggcgcgctggcttccgccttgacccgaatttgttttccttccgttaaaaaaatggttttccttttcttaaaaaggaaatagtttgttttttaagtctgtgtattaggattattacacttgaattttggtatatgtgtaggataatttactgcatgtttataatagagttgtactatagatgaaataacccaatttttggtataattcgtgtttggttggaggtcaaaataacaggttattttgtgaagaaaaaactccgtagtatagtaccatatccatcatgaatacacatactgcctagacgagtgattaggatgaatccatgttatattcctcaaaataatataaaccacttgatcttatgatcttatccaatctgttcatataaactggagatataagatggtgcatttcccttttgatttcttttgttgacggccatgagataggttgcatccactgcatttatattttggaccaatacaatgcacctattgatacatggggacagctcaactaaccatgatgcaaaatgctggttggtgaccagttcttggcattatgataatgataggattaaaaaaaacagtgcaatgtctcggaaagaaaccatgacaaagggtacatgttgcattccagtttctaatgataaaattatgtgccagcaattcaaaaatcatgcgtgttccctacgcaccattctttgcaataaacaagtgcatgcacaatatgattgtgctaaggttcaagaacttgttgcagtggctaagcttggcgcgcctcgcgaccacctttaattaagtgaagagcaggagcttgcatgcctgcaggctctagaggatcccccctcagaagaccagagggctattgagacttttcaacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacaggcttcttgagatccttcaacaattaccaacaacaacaaacaacaaacaacattacaattactatttacaattacagtcgactctagaggatccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaaagcggccgcccggctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgatgataagctgtcaaacatgagaattcctttcgtcgacccacgtgttgctgaggtatttaaatcgcctcttcctcttccctcacgcctcttcctcttccctcacgcctcttcctcttccctcacgcctcttcctcttccctcatctagagtcgaccgcaagacccttcctctatataaggaagttcatttcatttggagaggacacgctcgagtggccaccatttaaatcaactagggatatcacaagtttgtacaaaaaagcaggcttcacaatgttacgtcctgtagaaaccccaacccgtgaaatcaaaaaactcgacggcctgtgggcattcagtctggatcgcgaaaactgtggaattggtcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaaacccagctttc

SEQ ID NO: 36 - DNA, 22 bp, Artificial Sequence
  stress inducible element
ggaggaggaggaggaggaggag

SEQ ID NO: 37 - DNA, 14 bp, Artificial Sequence
  stress inducible element
wggwggagnrdrrr

SEQ ID NO: 38 - DNA, 30 bp, Artificial Sequence
  repitition motif comprising stress inducible element
aggaggaggaggaggaggaggaggaggagg

SEQ ID NO: 39 - DNA, 198 bp, Artificial Sequence
  Artificial Promoter
aggaggaggaggaggaggaggaggaggaggaggaggaggaggaggaggaggaggaggaggaggaggaggaggaggaggaggaggaggaggaggaggaggaggaggaggaggaggaggaggtctagagtcgaccgcaagacccttcctctatataaggaagttcatttcatttggagaggacacgctcgagtggccacc

SEQ ID NO: 40 - DNA, 13702 bp, Artificial Sequence
  Expression vector
ttgtacaaagtggtgatatcacaagcccgggcggtcttctagggataacagggtaattatatccctctagatcacaagcccgggcggtcttctacgatgattgagtaataatgtgtcacgcatcaccatgggtggcagtgtcagtgtgagcaatgacctgaatgaacaattgaaatgaaaagaaaaaaagtactccatctgttccaaattaaaattggttttaaccttttaataggtttatacaataattgatatatgttttctgtatatgtctaatttgttatcatccgggcggtcttctagggataacagggtaattatatccctctagacaacacacaacaaataagagaaaaaacaaataatattaatttgagaatgaacaaaaggaccatatcattcattaactcttctccatccacttccatttcacagttcgatagcgaaaaccgaataaaaaacacagtaaattacaagcacaacaaatggtacaagaaaaacagttttcccaatgccataatactcgactcgagttcctgcaggtaccaaaagcttagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtatgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgcttagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattcaagagctcaaggatcctaactataacggtcctaaggtagcgaaggcgcgccgaattcgaggggatcgagcccctgctgagcctcgacatgttgtcgcaaaattcgccctggacccgcccaacgatttgtcgtcactgtcaaggtttgacctgcacttcatttggggcccacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgagcgttattagttcgccgctcggtgtgtcgtagatactagcccctggggcacttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcagggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccacatgatcaaacgttttgaggacgcgagaggattcgattcgacgacgagagcctcgcgagattggggagaaatttttcgggggtggagctgatgcgaggagaggagatgagggggctggtatttatggcggttgggtggtgggaggagtcccgtgccgtgacgtctccgtctgcttggagaatccgccacgctgaaaccaccgcggtttccgggaagacgaggcgggcgagcgagcggttgggaaatttcgagaagatgccgtttgtctccgtttggtacacgtctcgttgatttttttttagtgaattacgctttggaccacattttattatctaagggtgtgtttggttgtaagccacactttgccacagtttgccacgcctaaggttaggcaaatttgacaggtgtttggttgtagccacagttgtggcaagatttccctctaacaaattaagtcccacgtgtcaatggctcaaaaaagtgtggcaagattcccttaggcttagtaagttgtggctaacaatttgatcacctcaccttagacaaggtgtggcaacttttgttggcaagtaatggtaaagtatggctgggaaccaaacagcccctaagttttactttggactacctttaaacatatcttttcactttgaactagataaatttgctattgttgcgatttggattttttttttctcgtgcaatcaacgaccttaaacacatcagctctagtatacggccgatctcctctatatatggttcatatgtttgccgaaagggaagttagacatgacgaaaagttgttcatggtagtccaaaccacaacccggcccaatttgaaaagataggtttaagggtggtccaaattgaaacttgggtaataaaaggtggatcaaagtgcaatttacttttttttactgtaatttcttctggctggtttgttggtcgccgttaggaccgggtgacgccgtcaaccccgcgcctccgtattcgctgacgtggggtggcgcgctggcttccgccttgacccgaatttgttttccttccgttaaaaaaatggttttccttttcttaaaaaggaaatagtttgttttttaagtctgtgtattaggattattacacttgaattttggtatatgtgtaggataatttactgcatgtttataatagagttgtactatagatgaaataacccaatttttggtataattcgtgtttggttggaggtcaaaataacaggttattttgtgaagaaaaaactccgtagtatagtaccatatccatcatgaatacacatactgcctagacgagtgattaggatgaatccatgttatattcctcaaaataatataaaccacttgatcttatgatcttatccaatctgttcatataaactggagatataagatggtgcatttcccttttgatttcttttgttgacggccatgagataggttgcatccactgcatttatattttggaccaatacaatgcacctattgatacatggggacagctcaactaaccatgatgcaaaatgctggttggtgaccagttcttggcattatgataatgataggattaaaaaaaacagtgcaatgtctcggaaagaaaccatgacaaagggtacatgttgcattccagtttctaatgataaaattatgtgccagcaattcaaaaatcatgcgtgttccctacgcaccattctttgcaataaacaagtgcatgcacaatatgattgtgctaaggttcaagaacttgttgcagtggctaagcttggcgcgcctcgcgaccacctttaattaagtgaagagcaggagcttgcatgcctgcaggctctagaggatcccccctcagaagaccagagggctattgagacttttcaacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacaggcttcttgagatccttcaacaattaccaacaacaacaaacaacaaacaacattacaattactatttacaattacagtcgactctagaggatccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaaagcggccgcccggctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgatgataagctgtcaaacatgagaattcctttcgtcgacccacgtgttgctgaggtatttaaataggaggaggaggaggaggaggaggaggaggaggaggaggaggaggaggaggaggaggaggaggaggaggaggaggaggaggaggaggaggaggaggaggaggaggaggaggaggaggaggtctagagtcgaccgcaagacccttcctctatataaggaagttcatttcatttggagaggacacgctcgagtggccaccatttaaatcaactagggatatcacaagtttgtacaaaaaagcaggcttcacaatgttacgtcctgtagaaaccccaacccgtgaaatcaaaaaactcgacggcctgtgggcattcagtctggatcgcgaaaactgtggaattggtcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaaacccagctttc

SEQ ID NO: 41 - DNA, 278 bp, Artificial Sequence
  Artificial Promoter
gtttgttggagtcgcaatcagtttgttggagtcgcaatcagtttgttggagtcgcaatcagtttgttggagtcgcaatcaccacgcctctctccacctcgcccacgcacaccacgcctctctccacctcgcccacgcacaccacgcctctctccacctcgcccacgcacaccacgcctctctccacctcgcccacgcacatctagagtcgaccgcaagacccttcctctatataaggaagttcatttcatttggagaggacacgctcgagtggccacc

SEQ ID NO: 42 - DNA, 278 bp, Artificial Sequence
  Artificial Promoter
cgcctcttcctcttccctcacgcctcttcctcttccctcacgcctcttcctcttccctcacgcctcttcctcttccctcaccacgcctctctccacctcgcccacgcacaccacgcctctctccacctcgcccacgcacaccacgcctctctccacctcgcccacgcacaccacgcctctctccacctcgcccacgcacatctagagtcgaccgcaagacccttcctctatataaggaagttcatttcatttggagaggacacgctcgagtggccacc

SEQ ID NO: 43 - DNA, 398 bp, Artificial Sequence
  Artificial Promoter
gcaatcgggacccacgtcgcactctaccccgcaatcgggacccacgtcgcactctaccccgcaatcgggacccacgtcgcactctaccccgcaatcgggacccacgtcgcactctacccctttcttgactttggaccctgccccactatctttcttgactttggaccctgccccactatctttcttgactttggaccctgccccactatctttcttgactttggaccctgccccactatccaaaatgtgtgcacaacaaccaaaatgtgtgcacaacaaccaaaatgtgtgcacaacaaccaaaatgtgtgcacaacaactctagagtcgaccgcaagacccttcctctatataaggaagttcatttcatttggagaggacacgctcgagtggccacc

SEQ ID NO: 44 - DNA, 583 bp, Oryza sativa
  Intron
gtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcag

SEQ ID NO: 45 - DNA, 872 bp, Artificial Sequence
  Artificial Promoter
gtttgttggagtcgcaatcagtttgttggagtcgcaatcagtttgttggagtcgcaatcagtttgttggagtcgcaatcaccacgcctctctccacctcgcccacgcacaccacgcctctctccacctcgcccacgcacaccacgcctctctccacctcgcccacgcacaccacgcctctctccacctcgcccacgcacatctagagtcgaccgcaagacccttcctctatataaggaagttcatttcatttggagaggacacgctcctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagatgcaagatgtac

SEQ ID NO: 46 - DNA, 872 bp, Artificial Sequence
  Artificial Promoter
cgcctcttcctcttccctcacgcctcttcctcttccctcacgcctcttcctcttccctcacgcctcttcctcttccctcaccacgcctctctccacctcgcccacgcacaccacgcctctctccacctcgcccacgcacaccacgcctctctccacctcgcccacgcacaccacgcctctctccacctcgcccacgcacatctagagtcgaccgcaagacccttcctctatataaggaagttcatttcatttggagaggacacgctcctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagatgcaagatgtac

SEQ ID NO: 47 - DNA, 992 bp, Artificial Sequence
  Artificial Promoter
gcaatcgggacccacgtcgcactctaccccgcaatcgggacccacgtcgcactctaccccgcaatcgggacccacgtcgcactctaccccgcaatcgggacccacgtcgcactctacccctttcttgactttggaccctgccccactatctttcttgactttggaccctgccccactatctttcttgactttggaccctgccccactatctttcttgactttggaccctgccccactatccaaaatgtgtgcacaacaaccaaaatgtgtgcacaacaaccaaaatgtgtgcacaacaaccaaaatgtgtgcacaacaactctagagtcgaccgcaagacccttcctctatataaggaagttcatttcatttggagaggacacgctcctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagatgcaagatgtac

SEQ ID NO: 48 - DNA, 752 bp, Artificial Sequence
  Artificial Promoter
tattttgtgagagagctccatattttgtgagagagctccatattttgtgagagagctccatattttgtgagagagctccatctagagtcgaccgcaagacccttcctctatataaggaagttcatttcatttggagaggacacgctcctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagatgcaagatgtac

SEQ ID NO: 49 - DNA, 792 bp, Artificial Sequence
  Artificial Promoter
gcaatcgggacccacgtcgcactctaccccgcaatcgggacccacgtcgcactctaccccgcaatcgggacccacgtcgcactctaccccgcaatcgggacccacgtcgcactctacccctctagagtcgaccgcaagacccttcctctatataaggaagttcatttcatttggagaggacacgctcctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagatgcaagatgtac

SEQ ID NO: 50 - DNA, 752 bp, Artificial Sequence
  Artificial Promoter
gtttgttggagtcgcaatcagtttgttggagtcgcaatcagtttgttggagtcgcaatcagtttgttggagtcgcaatcatctagagtcgaccgcaagacccttcctctatataaggaagttcatttcatttggagaggacacgctcctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagatgcaagatgtac

SEQ ID NO: 51 - DNA, 792 bp, Artificial Sequence
  Artificial Promoter
ccacgcctctctccacctcgcccacgcacaccacgcctctctccacctcgcccacgcacaccacgcctctctccacctcgcccacgcacaccacgcctctctccacctcgcccacgcacatctagagtcgaccgcaagacccttcctctatataaggaagttcatttcatttggagaggacacgctcctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagatgcaagatgtac

SEQ ID NO: 52 - DNA, 752 bp, Artificial Sequence
  Artificial Promoter
caaaatgtgtgcacaacaaccaaaatgtgtgcacaacaaccaaaatgtgtgcacaacaaccaaaatgtgtgcacaacaactctagagtcgaccgcaagacccttcctctatataaggaagttcatttcatttggagaggacacgctcctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagatgcaagatgtac

SEQ ID NO: 53 - DNA, 792 bp, Artificial Sequence
  Artificial Promoter
tttcttgactttggaccctgccccactatctttcttgactttggaccctgccccactatctttcttgactttggaccctgccccactatctttcttgactttggaccctgccccactatctctagagtcgaccgcaagacccttcctctatataaggaagttcatttcatttggagaggacacgctcctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagatgcaagatgtac

SEQ ID NO: 54 - DNA, 752 bp, Artificial Sequence
  Artificial Promoter
cgcctcttcctcttccctcacgcctcttcctcttccctcacgcctcttcctcttccctcacgcctcttcctcttccctcatctagagtcgaccgcaagacccttcctctatataaggaagttcatttcatttggagaggacacgctcctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagatgcaagatgtac

SEQ ID NO: 55 - DNA, 792 bp, Artificial Sequence
  Artificial Promoter
aggaggaggaggaggaggaggaggaggaggaggaggaggaggaggaggaggaggaggaggaggaggaggaggaggaggaggaggaggaggaggaggaggaggaggaggaggaggaggaggtctagagtcgaccgcaagacccttcctctatataaggaagttcatttcatttggagaggacacgctcctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagatgcaagatgtac

(19) United States
(12) Patent Application Publication
(10) Pub. No.: US 2015/0135368 A1
(43) Pub. Date: May 14, 2015

(54) Expression Cassettes for Stress-Induced Gene Expression in Plants

(71) Applicant: BASF Plant Science Company GmbH, Ludwigshafen (DE)

(72) Inventors: Anke Eisenmann, Freiburg (DE); Lorenz Bülow, Braunschweig (DE); Yves Hatzfeld, Lille (FR); Zhongyi Zhou, Balegem (BE)

(73) Assignee: BASFPlant Scence Company GmbH, Ludwigshafen (DE)

(21) Appl. No.: 14/382,039

(22) PCT Filed: Feb. 28, 2013
(86) PCT No.: PCT/IB2013/051591

§ 371 Date: Aug. 29, 2014

Related U.S. Application Data
(60) Provisional application No. 61/605,766, filed on Mar. 2, 2012.
(60) Provisional application No. 61/605,779, filed on Mar. 2, 2012.
(60) Provisional application No. 61/605,780, filed on Mar. 2, 2012.
(60) Provisional application No. 61/606,493, filed on Mar. 5, 2012.
(60) Provisional application No. 61/606,491, filed on Mar. 5, 2012.
(60) Provisional application No. 61/606,496, filed on Mar. 5, 2012.
(60) Provisional application No. 61/607,006, filed on Mar. 6, 2012.
(60) Provisional application No. 61/607,020, filed on Mar. 6, 2012.

(30) Foreign Application Priority Data
Mar. 2, 2012 (EP) 12157822.3
Mar. 2, 2012 (EP) 12157854.6
Mar. 2, 2012 (EP) 12157862.9
Mar. 5, 2012 (EP) 12158065.8
Mar. 5, 2012 (EP) 12158067.4
Mar. 5, 2012 (EP) 12158068.2
Mar. 6, 2012 (EP) 12158169.8
Mar. 6, 2012 (EP) 12158173.0

(51) Int. Cl. C12N 15/82

(52) U.S. Cl. CPC C12N 15/8237
USPC 800/298; 536/24.1; 435/320.1; 435/419; 435/6.12

(57) ABSTRACT

The invention relates to promoters for stress-induced expression in plants as well as to methods for rendering any given promoter into a stress-inducible promoter. The invention further relates to promoter sequences produced with such methods and their use for stress-induced gene expression.

This application claims priority of applications with number U.S. 61/605,766, U.S. 61/605,779, U.S. 61/605,780, EP 12157822.3, EP 12157854.6, EP 12157862.9, U.S. 61/606,491, U.S. 61/606,493, U.S. 61/606,496, EP 12158065.8, EP 12158067.4, EP 12158068.2, U.S. 61/607,006, U.S. 61/607,020, EP 12158169.8 and EP 12158173.0, all of which are incorporated by reference in their entirety.

### FIELD OF THE INVENTION

The invention relates to promoters for stress-induced expression in plants as well as to methods for rendering any given promoter into a stress-inducible promoter. The invention further relates to promoter sequences and molecules produced with such methods and their use for stress-induced gene expression.

### BACKGROUND OF THE INVENTION

Manipulation of plants to alter and/or improve phenotypic characteristics (such as productivity, quality or stress resistance) requires the expression of heterologous genes in plant tissues. Such genetic manipulation relies on the availability of a means to drive and to control gene expression as required. For example, genetic manipulation relies on the availability and use of suitable promoters which are effective in plants and which regulate gene expression so as to give the desired effect(s) in the transgenic plant. For numerous applications in plant biotechnology a tissue-specific or developmental-specific expression profile is advantageous, since beneficial effects of expression in one tissue or timepoint may have disadvantages in others. For example, promoters driving drought-inducible expression are of importance for expression of genes involved in improved drought resistance in plants. It is advantageous to have the choice of a variety of different promoters so that the most suitable promoter may be selected for a particular gene, construct, cell, tissue, plant or environment. Moreover, the increasing interest in transforming plants with multiple plant transcription units (PTU) and the potential problems associated with using common regulatory molecules for these purposes merit having a variety of promoter molecules available.

There is, therefore, a constant need in the art for the identification of novel molecules that can be used for expression of selected transgenes in economically important plants. It is thus an objective of the present invention to provide new and alternative expression cassettes for stress-induced expression of transgenes. The objective is solved by the present invention.

### DETAILED DESCRIPTION OF THE INVENTION

A first embodiment of the invention is a recombinant promoter molecule for regulating stress-induced expression comprising at least one stress-induction element (SIE) or the complement or reverse complement thereof, wherein the at least one SIE comprises a sequence as defined by any of the matrices in any one of tables 3 to 10, wherein the matrix similarity is at least 0.8 and the core similarity is at least 0.75, wherein said recombinant promoter molecule is functionally linked to or comprising a minimal promoter heterologous to the at least one SIE.

SIEs represent binding or interacting sites for proteins and/or nucleic acids, for example DNA binding proteins or DNA binding nucleic acid molecules. SIEs are sequence motifs involved in stress-induced gene expression defined by a highly conserved core sequence of approximately 4 to 6 nucleotide positions surrounded by a conserved matrix sequence of in total up to 27 nucleotides within the plus or minus strand of the SIE, which is able of interacting with proteins or nucleic acids, for example DNA binding proteins or DNA binding nucleic acid molecules. Preferably the 4 to 6 nucleotide positions of the core sequence are consecutive nucleotide positions. The conserved matrix sequence allows some variability in the sequence without loosing its ability to be bound by or to interact with the proteins or nucleic acid molecules, for example DNA binding proteins or nucleic acid molecules.

One way to describe DNA binding protein or nucleic acid binding sites is by nucleotide or position weight matrices (NWM or PWM) (for review see Stormo, 2000). A weight matrix pattern definition is superior to a simple IUPAC consensus sequence as it represents the complete nucleotide distribution for each single position. It also allows the quantification of the similarity between the weight matrix and a potential DNA binding protein or nucleic acid binding site detected in the sequence (Cartharius et al. 2005).

The “core sequence” of a matrix is defined as the 4, 5 or 6 highest conserved positions of the matrix, preferably the 4, 5 or 6 highest conserved consecutive positions of the matrix.

The core similarity is calculated as described here and in the papers related to MatInspector (Cartharius K, et al. (2005) Bioinformatics 21; Cartharius K (2005), DNA Press; Quandt K, et al (1995) Nucleic Acids Res. 23).

The maximum core similarity of 1.0 is only reached when the highest conserved bases of a matrix match exactly in the sequence. More important than the core similarity is the matrix similarity which takes into account all bases over the whole matrix length. The matrix similarity is calculated as described here and in the MatInspector paper (Quandt K, et al (1995) Nucleic Acids Res. 23). A perfect match to the matrix gets a score of 1.00 (each sequence position corresponds to the highest conserved nucleotide at that position in the matrix), a “good” match to the matrix has a similarity of >=0.80.

Mismatches in highly conserved positions of the matrix decrease the matrix similarity more than mismatches in less conserved regions.

In one embodiment the SIEs have a sequence as defined by the IUPAC string in any of tables 3 to 10. In another embodiment, the sequences of the SIEs are defined as the matrix described in any of tables 3 to 10, wherein core and matrix similarity of a matching sequence are calculated as described in Quandt et al (1995, NAR 23 (23) 4878-4884) in equation 2 and 3 on page 4879 right column, wherein the matrix similarity is at least 0.8, preferably the matrix similarity is at least 0.85, more preferably the matrix similarity is at least 0.9, even more preferably the matrix similarity is at least 0.95. In a most preferred embodiment the matrix similarity is at least 1.0. In one embodiment the core similarity is at least 0.75, preferably the core similarity is at least 0.8 for example 0.85, more preferably the core similarity is at least 0.9, even more preferably the core similarity is at least 0.95. In a most preferred embodiment the core similarity is at least 1.0.

The core sequences of the SIE sequences are defined in tables 3 to 10.

Stresses that may induce the expression derived from the promoter of the invention may be any biotic or abiotic stress. The inducing biotic stress may be wounding or pest infestation on the plant such as insects or fungae. Another exemplary biotic stress is bacterial or viral infection. Abiotic stresses that may lead to induction of the expression derived from the promoter of the invention are for example drought, flooding, heat, cold, low or high light. In a preferred embodiment the abiotic stress is drought, heat or hight light. In a most preferred embodiment the expression derived from the promoter of the invention is drought stress induced expression.

The recombinant promoter of the invention may comprise 1, 2, 3, 4, 5, 6, 7, 8, 9, 10, 11, 12 or more, for example 16 or 20 SIEs of the invention as defined herein. Preferably it comprises at least 4 or a multitude of 4 SIEs of the invention. More preferably the recombinant promoter comprises at least 4 copies of the same SIE of the invention or a multitude of 4 copies of the same SIE of the invention or a multitude of 4 copies each of various SIEs of the invention. In one embodiment the SIEs are located relative to each other in a way that the distance between the start of one SIE and the start of another SIE are separated by a number of nucleotides divisible by 10. Preferably the distance between the start of one SIE and the start of the adjacent SIE divisible by 10 is less than 1000, preferably less than 500, more preferably less than 100, for example less than 50 or less than 40 nucleotides, most preferably the distance is the lowest possible distance. Lowest possible distance means the distance between the start of two adjacent SIE is only the length of the respective SIE rounded up to the next number divisible by 10. For example, the lowest possible distance between the start of two adjacent SIE, the first SIE consisting of 12 bases, is 20 bases, the distance between the start of two adjacent SIE, the first consisting of 25 bases, is 30 bases.

In case the SIE itself consists of 10 or a multitude of 10 bases, the SIEs may be located directly adjacent to each other.

In another embodiment the recombinant promoter of the invention comprises 4 SIEs, preferably the same SIE that are located relative to each other in a way that the distance between the start of one SIE and the start of the adjacent SIE is 10 or a multitude of 10 bases. In case the SIE itself consists of 10 or a multitude of 10 bases, the SIEs may be located directly adjacent to each other. Preferably the distance between the start of one SIE and the start of the adjacent SIE divisible by 10 is less than 1000, preferably less than 500, more preferably less than 100, for example less than 50 or less than 40, most preferably the distance is the lowest possible distance.

In the event the promoter of the invention comprises more than one block of 4 SIEs, the start of a respective block of 4 SIEs has preferably a distance of a multitude of 10 bases to the start of another block of 4 SIEs. Preferably the distance between the start of the first SIE in the first block and the start of the first SIE in the adjacent block divisible by 10 is less than 1000, preferably less than 500, more preferably less than 100, for example less than 50, most preferably the distance is the lowest possible distance.

The block of 4 SIEs may comprise the same SIE or various SIEs. In the event the promoter of the invention comprises more than one block of SIEs, it may comprise several copies of the same block of SIEs or various blocks of SIEs.

For further illustration, a SIE may be comprised in a nucleotide sequence consisting of 10 or a multitude of 10 bases, said nucleotide sequence defined by A, B, C, D etc. A block may consist of any number and any combination of A, B, C and/or D. Preferably a block consists of 4 SIEs, preferably it consists of AAAA, BBBB, CCCC or DDDD. It may in another embodiment consist of ABAB, AABB, ABCD or any variation of A, B, C or D etc.

The promoter of the invention may comprise further motifs that are involved in stress induced expression in one or more copies. The motifs may already be present in the promoter in which the SIEs of the invention or the block(s) of 4 SIEs are introduced or may be introduced as additional recombinant SIEs or block(s) of 4 SIEs together with the SIEs of the invention. The SIEs already present in the promoter may be homologous or heterologous to the minimal promoter to which they are functionally linked. By introducing the SIEs of the invention into a promoter naturally comprising stress induction elements the degree of stress induction of a given stress-inducible promoter may be increased.

For example, the recombinant promoter of the invention may comprise 2 or more different of the SIEs of the invention as defined in Tables 3 to 10, preferably it comprises at least 4 copies each of at least 1, 2 or more of the SIEs of the invention, for example at least 4 copies each of at least 3, 4, 5 or more of the SIEs of the invention. Preferred combinations of SIEs are given in Table 11.

The SIEs of the invention may be functionally linked to any minimal promoter heterologous to the SIEs, such as the 35S minimal promoter (Odell et al., 1985, nature 313, 810-812) or any other heterologous minimal promoter well known to a person skilled in the art. The minimal promoter may be derived from a virus, prokaryoic or eukaryotic organism, for example from a bacterium, fungi, animal or a plant, for example a monocotyledonous or dicotyledonous plant. The minimal promoter may consist of the minimal promoter or may be comprised in a promoter capable to drive expression in a plant. Introduction of at least one or more SIE into such promoter renders the promoter into a stress-inducible promoter. Any promoter functional in a plant of any specificity with the exception of stress-repressed promoters may by introduction of at least one SIE converted into a stress-inducible promoter. In a preferred embodiment, the introduction of the at least one SIE does not alter the specificity of the promoter. For example a leaf-specific promoter would upon introduction of at least one SIE be induced by stress in leaves but not necessarily in other tissues of the plant.

In a further embodiment of the invention, any stress inducible promoter comprising at least one SIE may be rendered into a non stress-inducible promoter by deleting or mutating at least one, preferably all SIEs in the respective promoter. Thereby a promoter, which is for example stress-induced in a specific tissue, may be turned into a tissue specific promoter which is not stress regulated.

A further embodiment of the invention is the recombinant promoter molecule for regulating stress-inducible expression as described above wherein the at least one SIE is defined by any of SEQ ID NO: 1, 2, 3, 6, 7, 8, 11, 12, 13, 16, 17, 18, 21, 22, 23, 26, 27, 28, 31, 32, 33, 36, 37 or 38 or the complement or reverse complement thereof.

Preferably, the recombinant promoter molecule of the invention for regulating stress-inducible expression in a plant or part thereof comprises a combination of various SIEs, preferably a combination of blocks of 4 copies each of various SIEs of the invention, more preferably it comprises at least two, three or four different SIEs, preferably a combination of blocks of 4 copies each of at least two, three of four different SIEs. Preferred combinations of SIEs are given in Table 11. Most preferably the recombinant promoter of the invention comprising various SIEs has the sequence of SEQ ID NO: 41, 42, 43 or variants thereof as described below.

Recombinant promoter molecules as described above wherein the recombinant promoter molecule comprising said at least one SIE regulates stress-induced expression of heterologous nucleic acid molecules to which the recombinant promoter molecule is functionally linked is also one embodiment of the invention. In a preferred embodiment, the expression of the nucleic acid molecule functionally linked to a recombinant promoter of the invention is hardly, more preferably not detectable before induction. In case it is detectable before induction, the expression is induced at least by at least 10%, 20% or 30%, for example at least 50%, more preferably by at least 75%, for example at least 100%, even more preferably it is induced at least 3 fold, 5 fold, 10 fold or 50 fold. It may also be induced at least 100 fold. Recombinant promoters showing no expression before stress induction exhibit detectable expression after stress induction.

A further embodiment of the invention is a recombinant promoter molecule, for example an isolated recombinant promoter molecule, as described above selected from the group consisting of
a) the nucleic acid molecules having a sequence as defined in any of SEQ ID NO: 4, 9, 14, 19, 24, 29, 34, 39, 41, 42, 43, 45, 46, 47, 48, 49, 50, 51, 52, 53, 54 or 55 or
b) a nucleic acid molecule having a sequence with an identity of at least 60%, 65% or 70%, preferably at least 75%, 80% or 85%, more preferably at least 90% or 95%, even more preferably at least 96% or 97%, most preferably at least 98% or 99% to any of SEQ ID NO: 4, 9, 14, 19, 24, 29, 34, 39, 41, 42, 43, 45, 46, 47, 48, 49, 50, 51, 52, 53, 54 or 55, or
c) a fragment of 100 or more, for example 150 or more, preferably 160 or more, more preferably 170 or more, even more preferably 180, 190 or 200 or more consecutive bases of a nucleic acid molecule of a) or b), or
d) a nucleic acid molecule of 50 or more, preferably 100 or more, more preferably 150 or more, even more preferably 180 or more, 190 or more or 200 or more nucleotides, hybridizing under conditions equivalent to hybridization in 7% sodium dodecyl sulfate (SDS), 0.5 M NaPO4, 1 mM EDTA at 50° C. with washing in 2×SSC, 0.1% SDS at 50° C., more desirably in 7% SDS, 0.5 M NaPO4, 1 mM EDTA at 50° C. with washing in 1×SSC, 0.1% SDS at 65° C., even more desirably in 7% SDS, 0.5 M NaPO4, 1 mM EDTA at 50° C. with washing in 0.1×SSC, 0.1% SDS at 65° C. to a nucleic acid molecule comprising 50 or more, preferably 100 or more, more preferably 150 or more consecutive nucleotides of the promoter described by any of SEQ ID NO: 4, 9, 14, 19, 24, 29, 34, 39, 41, 42, 43, 45, 46, 47, 48, 49, 50, 51, 52, 53, 54 or 55 or the complement thereof,
e) a nucleic acid molecule which is the complement or reverse complement of any of the previously mentioned nucleic acid molecules under a) to d),
   wherein the promoter molecules as defined under b) to e) cause stress-induced expression of heterologous nucleic acid molecules to which they are functionally linked. Preferably they cause at least 10%, for example at least 20%, more preferably at least 30%, for example at least 50%, even more preferably at least 75% of stress induced expression as the respective recombinant promoter defined in a), preferably the cause stress induced expression at least to the same extent as the respective promoter defined in a).

In one embodiment, the promoters as defined under b), c) and d) above comprise a minimal promoter.

A person skilled in the art is aware of methods for rendering a unidirectional to a bidirectional promoter and of methods to use the complement or reverse complement of a promoter sequence as defined above under e) for creating a promoter having the same promoter specificity as the original sequence. Such methods are for example described for constitutive as well as inducible promoters by Xie et al. (2001) Bidirectionalization of polar promoters in plants” nature biotechnology 19, pages 677-679. The authors describe that it is sufficient to add a minimal promoter to the 5′ end of any given promoter to receive a promoter controlling expression in both directions with same properties such as promoter specificity and strength.

Preferably the promoter nucleic acid molecules as defined above under b) to e) are functional stress-inducible promoters having at least 10%, preferably at least 20%, more preferably at least 30%, for example at least 50%, even more preferably at least 75% of the stress-inducible effect as the respective molecule as defined above under a). In a most preferred embodiment, the nucleic acid molecules as defined above under b) to e) have the same stress-inducible effect, hence induce expression under stress conditions to the same extend as the respective molecule defined under a).

A recombinant expression construct comprising one or more recombinant promoter molecules as defined above is a further embodiment of the invention. Preferably, the one or more recombinant promoter molecules comprised in the recombinant expression construct of the invention are functionally linked to a nucleic acid molecule to be expressed, the latter being heterologous to the recombinant promoter molecule to which it is functionally linked.

A recombinant expression vector comprising one or more recombinant expression constructs as described above or one or more recombinant promoter molecules as described above is a further one embodiment of the invention.

A transgenic cell, a transgenic plant or part thereof comprising one or more vectors as defined above, one or more recombinant expression constructs as defined above or one or more recombinant promoter molecules as described above is a further embodiment of the invention. Preferentially the transgenic cell, transgenic plant or part thereof as described above is selected or derived from the group consisting of bacteria, fungi, yeasts or plants.

A transgenic cell culture, transgenic seed, parts or propagation material derived from a transgenic cell or plant or part thereof as defined above comprising one or more vectors as defined above, one or more recombinant expression constructs as defined above or one or more recombinant promoter molecules as defined above is a further embodiment of the invention. In a preferred embodiment the transgenic cell or plant or part thereof is a dicotyledonous or monocotyledonous transgenic cell or plant or part thereof, more preferably it is a monocotyledonous transgenic cell or plant or part thereof.

The transgenic cell, transgenic plant or part thereof may be selected from the group consisting of bacteria, fungi, yeasts, or plant, insect or mammalian cells or plants. Preferred transgenic cells are bacteria, fungi, yeasts and plant cells. Preferred bacteria are Enterobacteria such as E. coli and bacteria of the genus Agrobacteria, for example Agrobacterium tumefaciens and Agrobacterium rhizogenes. Preferred plants are monocotyledonous or dicotyledonous plants for example monocotyledonous or dicotyledonous crop plants such as corn, soy, canola, cotton, potato, sugar beet, rice, wheat, sorghum, barley, musa, sugarcane, miscanthus and the like. Preferred crop plants are corn, rice, wheat, soy, canola, cotton or potato. Especially preferred dicotyledonous crop plants are soy, canola, cotton or potato. Especially preferred monocotyledonous crop plants are corn, especially zea mays, wheat, especially Triticum aestivum and Triticum durum and rice, especially Oryza sativa.

Transgenic parts as meant herein comprise all tissues and organs, for example leaf, stem and fruit as well as material that is useful for propagation and/or regeneration of plants such as cuttings, scions, layers, branches or shoots comprising the respective recombinant promoter molecule, recombinant expression construct or recombinant vector.

A method for the identification and isolation of promoter molecules for stress-induced expression comprising the steps of
A) identification of a promoter molecule in genomic DNA and
B) identification of promoters of A) that contain any of the matrices as defined in tables 3 to 10 or any of the SIEs as defined by SEQ ID NO: 1, 2, 3, 6, 7, 8, 11, 12, 13, 16, 17, 18, 21, 22, 23, 26, 27, 28, 31, 32, 33, 36, 37 or 38 using any computational SIE detection or IUPAC string matching sequence analysis tools and
C) isolation of at least 250 bp, preferably at least 300 bp, for example at least 400 bp more preferably at least 500 bp, for example 750 bp or 1000 bp of genomic DNA comprising said SIEs and a minimal promoter is another embodiment of the invention.

The promoter in the genomic DNA may be identified by any method known to a person skilled in the art, for example sequence determination of full-length cDNAs, computational predictions for example based on the prediction of coding sequences in genomic DNA sequences or the use of annotated databases for e.g. cDNAs or genomic DNA.

Identification of molecules in the group identified in A) that contain any of the SIEs or combinations thereof may for example be done with tools known to the skilled person, such as MatInspector of Genomatix Software GmbH, The MEME suite of the University of Queensland and University of Washington, or comparable tools. The isolation of the at least 250 bp, for example 300 bp, preferably at least 350 bp, for example 400 bp, more preferably at least 450 bp, for example 500 bp or 1000 bp may be done with recombinant methods known in the art such as PCR, restriction cloning or gene synthesis. The isolated stress-inducible promoter molecules may in subsequent steps be functionally linked to nucleic acid molecules to be expressed and/or other regulatory nucleic acid molecules such as 5″UTRs, terminators, NEENAs (WO2011/023537; WO2011/023539), introns and the like. A skilled person is aware of various methods for functionally linking two or more nucleic acid molecules. Such methods may encompass restriction/ligation, ligase independent cloning, recombineering, recombination or synthesis. Other methods may be employed to functionally link two or more nucleic acid molecules.

A method for providing stress-inducible promoter molecules comprising the step of functionally linking at least one SIE as defined by SEQ ID NO: 1, 2, 3, 6, 7, 8, 11, 12, 13, 16, 17, 18, 21, 22, 23, 26, 27, 28, 31, 32, 33, 36, 37 or 38 or combinations of thereof as defined above to a minimal promoter is a further embodiment of the invention.

Said minimal promoter may be comprised in a plant promoter, said plant promoter may be derived from a dicotyledonous or monocotyledonous plant. Preferentially it is derived from a monocotyledonous plant, more preferentially from a plant of the Oryza family, most preferably from the genus Oryza sativa.

A use of the promoter molecule defined above or the recombinant expression construct or recombinant vector as defined above for stress-induced expression in plants or parts thereof is another embodiment of the invention.

### DEFINITIONS

### Abbreviations

NEENA—nucleic acid expression enhancing nucleic acid, GFP—green fluorescence protein, GUS—beta-Glucuronidase, BAP—6-benzylaminopurine; 2,4-D—2,4-dichlorophenoxyacetic acid; MS—Murashige and Skoog medium; NAA—1-naphtaleneacetic acid; MES, 2-(N-morpholino-ethanesulfonic acid, IAA indole acetic acid; Kan: Kanamycin sulfate; GA3—Gibberellic acid; Timentin™: ticarcillin disodium/clavulanate potassium, microl: Microliter; SIE—stress-induction element.

It is to be understood that this invention is not limited to the particular methodology or protocols.

It is also to be understood that the terminology used herein is for the purpose of describing particular embodiments only, and is not intended to limit the scope of the present invention which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth. The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). As used herein, the word “or” means any one member of a particular list and also includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. For clarity, certain terms used in the specification are defined and used as follows:

Agricultural product: The term “Agricultural product” as used in this application means any harvestable product from a plant. The plant products may be, but are not limited to, foodstuff, feedstuff, food supplement, feed supplement, fiber, cosmetic or pharmaceutical product. Foodstuffs are regarded as compositions used for nutrition or for supplementing nutrition. Animal feedstuffs and animal feed supplements, in particular, are regarded as foodstuffs. Agricultural products may as an example be plant extracts, proteins, amino acids, carbohydrates, fats, oils, polymers such as starch or fibers, vitamins, secondary plant products and the like.

Antiparallel: “Antiparallel” refers herein to two nucleotide sequences paired through hydrogen bonds between complementary base residues with phosphodiester bonds running in the 5′-3′ direction in one nucleotide sequence and in the 3′-5′ direction in the other nucleotide sequence.

Antisense: The term “antisense” refers to a nucleotide sequence that is inverted relative to its normal orientation for transcription or function and so expresses an RNA transcript that is complementary to a target gene mRNA molecule expressed within the host cell (e.g., it can hybridize to the target gene mRNA molecule or single stranded genomic DNA through Watson-Crick base pairing) or that is complementary to a target DNA molecule such as, for example genomic DNA present in the host cell.

Coding region: As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′-side by the nucleotide triplet “ATG” which encodes the initiator methionine and on the 3′-side by one of the three triplets which specify stop codons (i.e., TAA, TAG, TGA). In addition to containing introns, genomic forms of a gene may also include sequences located on both the 5′- and 3′-end of the sequences which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers which control or influence the transcription of the gene. The 3′-flanking region may contain sequences which direct the termination of transcription, post-transcriptional cleavage and polyadenylation.

Complementary: “Complementary” or “complementarity” refers to two nucleotide sequences which comprise antiparallel nucleotide sequences capable of pairing with one another (by the base-pairing rules) upon formation of hydrogen bonds between the complementary base residues in the antiparallel nucleotide sequences. For example, the sequence 5′-AGT-3′ is complementary to the sequence 5′-ACT-3′. Complementarity can be “partial” or “total.” “Partial” complementarity is where one or more nucleic acid bases are not matched according to the base pairing rules. “Total” or “complete” complementarity between nucleic acid molecules is where each and every nucleic acid base is matched with another base under the base pairing rules. The degree of complementarity between nucleic acid molecule strands has significant effects on the efficiency and strength of hybridization between nucleic acid molecule strands. A “complement” of a nucleic acid sequence as used herein refers to a nucleotide sequence whose nucleic acid molecules show total complementarity to the nucleic acid molecules of the nucleic acid sequence.

Double-stranded RNA: A “double-stranded RNA” molecule or “dsRNA” molecule comprises a sense RNA fragment of a nucleotide sequence and an antisense RNA fragment of the nucleotide sequence, which both comprise nucleotide sequences complementary to one another, thereby allowing the sense and antisense RNA fragments to pair and form a double-stranded RNA molecule.

Endogenous: An “endogenous” nucleotide sequence refers to a nucleotide sequence, which is present in the genome of the untransformed plant cell.

Enhanced expression: “enhance” or “increase” the expression of a nucleic acid molecule in a plant cell are used equivalently herein and mean that the level of expression of the nucleic acid molecule in a plant, part of a plant or plant cell at a certain timepoint is higher than its expression in the plant, part of the plant or plant cell before this timepoint, for example before applying a enhancing stimulus such as stress. The term “enhanced” or “increased” as used herein are synonymous and means herein higher, preferably significantly higher expression of the nucleic acid molecule to be expressed. As used herein, an “enhancement” or “increase” of the level of an agent such as a protein, mRNA or RNA means that the level is increased relative to a substantially identical plant, part of a plant or plant cell grown under substantially identical conditions not exposed to the enhancing stimulus. As used herein, “enhancement” or “increase” of the level of an agent, such as for example a preRNA, mRNA, rRNA, tRNA, snoRNA, snRNA expressed by the target gene and/or of the protein product encoded by it, means that the level is increased 20% or more, for example 50% or more, preferably 100% or more, more preferably 3 fold or more, even more preferably 15 fold or more, most preferably 10 fold or more for example 20 fold relative to the timepoint before the enhancing stimulus or relative to an identical plant or part thereof not exposed to the stimulus such as stress, e.g. drought stress. The enhancement or increase can be determined by methods with which the skilled worker is familiar. Thus, the enhancement or increase of the nucleic acid or protein quantity can be determined for example by an immunological detection of the protein. Moreover, techniques such as protein assay, fluorescence, Northern hybridization, nuclease protection assay, reverse transcription (quantitative RT-PCR), ELISA (enzyme-linked immunosorbent assay), Western blotting, radioimmunoassay (RIA) or other immunoassays and fluorescence-activated cell analysis (FACS) can be employed to measure a specific protein or RNA in a plant or plant cell. Depending on the type of the induced protein product, its activity or the effect on the phenotype of the organism or the cell may also be determined. Methods for determining the protein quantity are known to the skilled worker. Examples, which may be mentioned, are: the micro-Biuret method (Goa J (1953) Scand J Clin Lab Invest 5:218-222), the Folin-Ciocalteau method (Lowry O H et al. (1951) J Biol Chem 193:265-275) or measuring the absorption of CBB G-250 (Bradford M M (1976) Analyt Biochem 72:248-254).

Expression: “Expression” refers to the biosynthesis of a gene product, preferably to the transcription and/or translation of a nucleotide sequence, for example an endogenous gene or a heterologous gene, in a cell. For example, in the case of a structural gene, expression involves transcription of the structural gene into mRNA and—optionally—the subsequent translation of mRNA into one or more polypeptides. In other cases, expression may refer only to the transcription of the DNA harboring an RNA molecule.

Expression construct: “Expression construct” as used herein mean a DNA sequence capable of directing expression of a particular nucleotide sequence in an appropriate part of a plant or plant cell, comprising a promoter functional in said part of a plant or plant cell into which it will be introduced, operatively linked to the nucleotide sequence of interest which is—optionally—operatively linked to termination signals. If translation is required, it also typically comprises sequences required for proper translation of the nucleotide sequence. The coding region may code for a protein of interest but may also code for a functional RNA of interest, for example RNAa, siRNA, snoRNA, snRNA, microRNA, ta-siRNA or any other noncoding regulatory RNA, in the sense or antisense direction. The expression construct comprising the nucleotide sequence of interest may be chimeric, meaning that one or more of its components are heterologous with respect to one or more of its other components. The expression construct may also be one, which is naturally occurring but has been obtained in a recombinant form useful for heterologous expression. Typically, however, the expression construct is heterologous with respect to the host, i.e., the particular DNA sequence of the expression construct does not occur naturally in the host cell and must have been introduced into the host cell or an ancestor of the host cell by a transformation event. The expression of the nucleotide sequence in the expression construct may be under the control of a seed-specific and/or seed-preferential promoter or of an inducible promoter, which initiates transcription only when the host cell is exposed to some particular external stimulus. In the case of a plant, the promoter can also be specific to a particular tissue or organ or stage of development.

Foreign: The term “foreign” refers to any nucleic acid molecule (e.g., gene sequence) which is introduced into the genome of a cell by experimental manipulations and may include sequences found in that cell so long as the introduced sequence contains some modification (e.g., a point mutation, the presence of a selectable marker gene, etc.) and is therefore distinct relative to the naturally-occurring sequence.

Functional linkage: The term “functional linkage” or “functionally linked” is to be understood as meaning, for example, the sequential arrangement of a regulatory element (e.g. a promoter) with a nucleic acid sequence to be expressed and, if appropriate, further regulatory elements (such as e.g., a terminator or a NEENA) in such a way that each of the regulatory elements can fulfill its intended function to allow, modify, facilitate or otherwise influence expression of said nucleic acid sequence to be expressed. As a synonym the wording “operable linkage” or “operably linked” may be used. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. The distance between the promoter sequence and the nucleic acid sequence to be expressed recombinantly is preferably less than 200 base pairs, especially preferably less than 100 base pairs, very especially preferably less than 50 base pairs. In a preferred embodiment, the nucleic acid sequence to be transcribed is located behind the promoter in such a way that the transcription start is identical with the desired beginning of the chimeric RNA of the invention. Functional linkage, and an expression construct, can be generated by means of customary recombination and cloning techniques as described (e.g., in Maniatis T, Fritsch E F and Sambrook J (1989) Molecular Cloning: A Laboratory Manual, 2nd Ed., Cold Spring Harbor Laboratory, Cold Spring Harbor (N.Y.); Silhavy et al. (1984) Experiments with Gene Fusions, Cold Spring Harbor Laboratory, Cold Spring Harbor (N.Y.); Ausubel et al. (1987) Current Protocols in Molecular Biology, Greene Publishing Assoc. and Wiley Interscience; Gelvin et al. (Eds) (1990) Plant Molecular Biology Manual; Kluwer Academic Publisher, Dordrecht, The Netherlands). However, further sequences, which, for example, act as a linker with specific cleavage sites for restriction enzymes, or as a signal peptide, may also be positioned between the two sequences. The insertion of sequences may also lead to the expression of fusion proteins. Preferably, the expression construct, consisting of a linkage of a regulatory region for example a promoter and nucleic acid sequence to be expressed, can exist in a vector-integrated form and be inserted into a plant genome, for example by transformation.

Gene: The term “gene” refers to a region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (downstream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

Genome and genomic DNA: The terms “genome” or “genomic DNA” is referring to the heritable genetic information of a host organism. Said genomic DNA comprises the DNA of the nucleus (also referred to as chromosomal DNA) but also the DNA of the plastids (e.g., chloroplasts) and other cellular organelles (e.g., mitochondria). Preferably the terms genome or genomic DNA is referring to the chromosomal DNA of the nucleus.

Heterologous: The term “heterologous” with respect to a nucleic acid molecule or DNA refers to a nucleic acid molecule which is operably linked to, or is manipulated to become operably linked to, a second nucleic acid molecule to which it is not operably linked in nature, or to which it is operably linked at a different location in nature. A heterologous expression construct comprising a nucleic acid molecule and one or more regulatory nucleic acid molecule (such as a promoter or a transcription termination signal) linked thereto for example is a constructs originating by experimental manipulations in which either a) said nucleic acid molecule, or b) said regulatory nucleic acid molecule or c) both (i.e. (a) and (b)) is not located in its natural (native) genetic environment or has been modified by experimental manipulations, an example of a modification being a substitution, addition, deletion, inversion or insertion of one or more nucleotide residues. Natural genetic environment refers to the natural chromosomal locus in the organism of origin, or to the presence in a genomic library. In the case of a genomic library, the natural genetic environment of the sequence of the nucleic acid molecule is preferably retained, at least in part. The environment flanks the nucleic acid sequence at least at one side and has a sequence of at least 50 bp, preferably at least 500 bp, especially preferably at least 1,000 bp, very especially preferably at least 5,000 bp, in length. A naturally occurring expression construct—for example the naturally occurring combination of a promoter with the corresponding gene—becomes a transgenic expression construct when it is modified by non-natural, synthetic “artificial” methods such as, for example, mutagenization. Such methods have been described (U.S. Pat. No. 5,565,350; WO 00/15815). For example a protein encoding nucleic acid molecule operably linked to a promoter, which is not the native promoter of this molecule, is considered to be heterologous with respect to the promoter. Preferably, heterologous DNA is not endogenous to or not naturally associated with the cell into which it is introduced, but has been obtained from another cell or has been synthesized. Heterologous DNA also includes an endogenous DNA sequence, which contains some modification, non-naturally occurring, multiple copies of an endogenous DNA sequence, or a DNA sequence which is not naturally associated with another DNA sequence physically linked thereto. Generally, although not necessarily, heterologous DNA encodes RNA or proteins that are not normally produced by the cell into which it is expressed.

Hybridization: The term “hybridization” as used herein includes “any process by which a strand of nucleic acid molecule joins with a complementary strand through base pairing” (J. Coombs (1994) Dictionary of Biotechnology, Stockton Press, New York). Hybridization and the strength of hybridization (i.e., the strength of the association between the nucleic acid molecules) is impacted by such factors as the degree of complementarity between the nucleic acid molecules, stringency of the conditions involved, the Tm of the formed hybrid, and the G:C ratio within the nucleic acid molecules. As used herein, the term “Tm” is used in reference to the “melting temperature.” The melting temperature is the temperature at which a population of double-stranded nucleic acid molecules becomes half dissociated into single strands. The equation for calculating the Tm of nucleic acid molecules is well known in the art. As indicated by standard references, a simple estimate of the Tm value may be calculated by the equation: Tm=81.5+0.41(% G+C), when a nucleic acid molecule is in aqueous solution at 1 M NaCl [see e.g., Anderson and Young, Quantitative Filter Hybridization, in Nucleic Acid Hybridization (1985)]. Other references include more sophisticated computations, which take structural as well as sequence characteristics into account for the calculation of Tm. Stringent conditions, are known to those skilled in the art and can be found in Current Protocols in Molecular Biology, John Wiley & Sons, N.Y. (1989), 6.3.1-6.3.6.

“Identity”: “Identity” when used in respect to the comparison of two or more nucleic acid or amino acid molecules means that the sequences of said molecules share a certain degree of sequence similarity, the sequences being partially identical.

To determine the percentage identity (homology is herein used interchangeably) of two amino acid sequences or of two nucleic acid molecules, the sequences are written one underneath the other for an optimal comparison (for example gaps may be inserted into the sequence of a protein or of a nucleic acid in order to generate an optimal alignment with the other protein or the other nucleic acid).

The amino acid residues or nucleic acid molecules at the corresponding amino acid positions or nucleotide positions are then compared. If a position in one sequence is occupied by the same amino acid residue or the same nucleic acid molecule as the corresponding position in the other sequence, the molecules are homologous at this position (i.e. amino acid or nucleic acid “homology” as used in the present context corresponds to amino acid or nucleic acid “identity”. The percentage homology between the two sequences is a function of the number of identical positions shared by the sequences (i.e. % homology=number of identical positions/total number of positions×100). The terms “homology” and “identity” are thus to be considered as synonyms.

For the determination of the percentage identity of two or more amino acids or of two or more nucleotide sequences several computer software programs have been developed. The identity of two or more sequences can be calculated with for example the software fasta, which presently has been used in the version fasta 3 (W. R. Pearson and D. J. Lipman, PNAS 85, 2444 (1988); W. R. Pearson, Methods in Enzymology 183, 63 (1990); W. R. Pearson and D. J. Lipman, PNAS 85, 2444 (1988); W. R. Pearson, Enzymology 183, 63 (1990)). Another useful program for the calculation of identities of different sequences is the standard blast program, which is included in the Biomax pedant software (Biomax, Munich, Federal Republic of Germany).

This leads unfortunately sometimes to suboptimal results since BLAST does not always include complete sequences of the subject and the query. Nevertheless as this program is very efficient it can be used for the comparison of a huge number of sequences. The following settings are typically used for such comparisons of sequences:

-p Program Name [String]; -d Database [String]; default=nr; -i Query File [File In]; default=stdin; -e Expectation value (E) [Real]; default=10.0; -m alignment view options: 0=pairwise; 1=query-anchored showing identities; 2=query-anchored no identities; 3=flat query-anchored, show identities; 4=flat query-anchored, no identities; 5=query-anchored no identities and blunt ends; 6=flat query-anchored, no identities and blunt ends; 7=XML Blast output; 8=tabular; 9 tabular with comment lines [Integer]; default=0; -o BLAST report Output File [File Out] Optional; default=stdout; -F Filter query sequence (DUST with blastn, SEG with others) [String]; default=T; -G Cost to open a gap (zero invokes default behavior) [Integer]; default=0; -E Cost to extend a gap (zero invokes default behavior) [Integer]; default=0; -X X dropoff value for gapped alignment (in bits) (zero invokes default behavior); blastn 30, megablast 20, tblastx 0, all others 15 [Integer]; default=0; -I Show GI's in deflines [T/F]; default=F; -q Penalty for a nucleotide mismatch (blastn only) [Integer]; default=−3; -r Reward for a nucleotide match (blastn only) [Integer]; default=1; -v Number of database sequences to show oneline descriptions for (V) [Integer]; default=500; -b Number of database sequence to show alignments for (B) [Integer]; default=250; -f Threshold for extending hits, default if zero; blastp 11, blastn 0, blastx 12, tblastn 13; tblastx 13, megablast 0 [Integer]; default=0; -g Perform gapped alignment (not available with tblastx) [T/F]; default=T; -Q Query Genetic code to use [Integer]; default=1; -D DB Genetic code (for tblast[nx] only) [Integer]; default=1; -a Number of processors to use [Integer]; default=1; -O SeqAlign file [File Out] Optional; -J Believe the query defline [T/F]; default=F; -M Matrix [String]; default=BLOSUM62; -W Word size, default if zero (blastn 11, megablast 28, all others 3) [Integer]; default=0; -z Effective length of the database (use zero for the real size) [Real]; default=0; -K Number of best hits from a region to keep (off by default, if used a value of 100 is recommended) [Integer]; default=0; -P 0 for multiple hit, 1 for single hit [Integer]; default=0; -Y Effective length of the search space (use zero for the real size) [Real]; default=0; -S Query strands to search against database (for blast[nx], and tblastx); 3 is both, 1 is top, 2 is bottom [Integer]; default=3; -T Produce HTML output [T/F]; default=F; -I Restrict search of database to list of GI's [String] Optional; -U Use lower case filtering of FASTA sequence [T/F] Optional; default=F; -y X dropoff value for ungapped extensions in bits (0.0 invokes default behavior); blastn 20, megablast 10, all others 7 [Real]; default=0.0; -Z X dropoff value for final gapped alignment in bits (0.0 invokes default behavior); blastn/megablast 50, tblastx 0, all others 25 [Integer]; default=0; -R PSI-TBLASTN checkpoint file [File In] Optional; -n MegaBlast search [T/F]; default=F; -L Location on query sequence [String] Optional; -A Multiple Hits window size, default if zero (blastn/megablast 0, all others 40 [Integer]; default=0; -w Frame shift penalty (OOF algorithm for blastx) [Integer]; default=0; -t Length of the largest intron allowed in tblastn for linking HSPs (0 disables linking) [Integer]; default=0.

Results of high quality are reached by using the algorithm of Needleman and Wunsch or Smith and Waterman. Therefore programs based on said algorithms are preferred. Advantageously the comparisons of sequences can be done with the program PileUp (J. Mol. Evolution., 25, 351 (1987), Higgins et al., CABIOS 5, 151 (1989)) or preferably with the programs “Gap” and “Needle”, which are both based on the algorithms of Needleman and Wunsch (J. Mol. Biol. 48; 443 (1970)), and “BestFit”, which is based on the algorithm of Smith and Waterman (Adv. Appl. Math. 2; 482 (1981)). “Gap” and “BestFit” are part of the GCG software-package (Genetics Computer Group, 575 Science Drive, Madison, Wis., USA 53711 (1991); Altschul et al., (Nucleic Acids Res. 25, 3389 (1997)), “Needle” is part of the The European Molecular Biology Open Software Suite (EMBOSS) (Trends in Genetics 16 (6), 276 (2000)). Therefore preferably the calculations to determine the percentages of sequence homology are done with the programs “Gap” or “Needle” over the whole range of the sequences. The following standard adjustments for the comparison of nucleic acid sequences were used for “Needle”: matrix: EDNAFULL, Gap_penalty: 10.0, Extend_penalty: 0.5. The following standard adjustments for the comparison of nucleic acid sequences were used for “Gap”: gap weight: 50, length weight: 3, average match: 10.000, average mismatch: 0.000.

For example a sequence, which is said to have 80% identity with sequence SEQ ID NO: 4 at the nucleic acid level is understood as meaning a sequence which, upon comparison with the sequence represented by SEQ ID NO: 4 by the above program “Needle” with the above parameter set, has a 80% identity. Preferably the homology is calculated on the complete length of the query sequence, for example SEQ ID NO: 4.

Induction of expression: the terms “induction of expression”, “induced expression” and the like mean that the level of expression of a nucleic acid molecule in a plant, part of a plant or plant cell at a specific timepoint is higher than its expression in the plant, part of the plant or plant cell before that timepoint, or compared to a reference plant that has not be exposed for example to a certain stimulus such as biotic or abiotic stress. The reference plant comprises the same construct but is not exposed to the expression inducing stimulus. The term “induced” as used herein means a higher, preferably significantly higher expression of the nucleic acid molecule to be expressed. As used herein, an “induction” of the level of an agent such as a protein, mRNA or RNA means that the level is increased relative to a substantially identical plant, part of a plant or plant cell grown under substantially identical conditions but not exposed to the expression inducing stimulus, or is increase relative to the expression of the same nucleic acid before the stimulus had been applied. As used herein, “induction” of the level of an agent, such as for example a preRNA, mRNA, rRNA, tRNA, snoRNA, snRNA expressed by the gene functionally linked to a promoter of the invention and/or of the protein product encoded by it, means that the level is increased by 20% or more, for example 50% or more, preferably 100% or more, more preferably 3 fold or more, even more preferably 15 fold or more, most preferably 10 fold or more for example 20 fold. “Induction” may also mean that the nucleic acid under control of the promoter of the invention is not expressed before the inducing stimulus is applied and hence the expression is only detectable after the inducing stimulus had been applied. The induction may not be detectable immediately after application of the inducing stimulus but may only detectable after a certain period of time, such as, for example after 10 minute or more, for example 30 minutes or after 1, 2 or 3 hours.

The induction can be determined by methods with which the skilled worker is familiar. Thus, the induction of the nucleic acid or protein quantity can be determined for example by an immunological detection of the protein. Moreover, techniques such as protein assay, fluorescence, Northern hybridization, nuclease protection assay, reverse transcription (quantitative RT-PCR), ELISA (enzyme-linked immunosorbent assay), Western blotting, radioimmunoassay (RIA) or other immunoassays and fluorescence-activated cell analysis (FACS) can be employed to measure a specific protein or RNA in a plant or plant cell. Depending on the type of the induced protein product, its activity or the effect on the phenotype of the organism or the cell may also be determined. Methods for determining the protein quantity are known to the skilled worker. Examples, which may be mentioned, are: the micro-Biuret method (Goa J (1953) Scand J Clin Lab Invest 5:218-222), the Folin-Ciocalteau method (Lowry O H et al. (1951) J Biol Chem 193:265-275) or measuring the absorption of CBB G-250 (Bradford M M (1976) Analyt Biochem 72:248-254).

Intron: refers to sections of DNA (intervening sequences) within a gene that do not encode part of the protein that the gene produces, and that is spliced out of the mRNA that is transcribed from the gene before it is exported from the cell nucleus. Intron sequence refers to the nucleic acid sequence of an intron. Thus, introns are those regions of DNA sequences that are transcribed along with the coding sequence (exons) but are removed during the formation of mature mRNA. Introns can be positioned within the actual coding region or in either the 5′ or 3′ untranslated leaders of the pre-mRNA (unspliced mRNA). Introns in the primary transcript are excised and the coding sequences are simultaneously and precisely ligated to form the mature mRNA. The junctions of introns and exons form the splice site. The sequence of an intron begins with GU and ends with AG. Furthermore, in plants, two examples of AU-AC introns have been described: the fourteenth intron of the RecA-like protein gene and the seventh intron of the G5 gene from Arabidopsis thaliana are AT-AC introns. Pre-mRNAs containing introns have three short sequences that are—beside other sequences—essential for the intron to be accurately spliced. These sequences are the 5′ splice-site, the 3′ splice-site, and the branchpoint. mRNA splicing is the removal of intervening sequences (introns) present in primary mRNA transcripts and joining or ligation of exon sequences. This is also known as cis-splicing which joins two exons on the same RNA with the removal of the intervening sequence (intron). The functional elements of an intron is comprising sequences that are recognized and bound by the specific protein components of the spliceosome (e.g. splicing consensus sequences at the ends of introns). The interaction of the functional elements with the spliceosome results in the removal of the intron sequence from the premature mRNA and the rejoining of the exon sequences. Introns have three short sequences that are essential—although not sufficient—for the intron to be accurately spliced. These sequences are the 5′ splice site, the 3′ splice site and the branch point. The branchpoint sequence is important in splicing and splice-site selection in plants. The branchpoint sequence is usually located 10-60 nucleotides upstream of the 3′ splice site.

Isogenic: organisms (e.g., plants), which are genetically identical, except that they may differ by the presence or absence of a heterologous DNA sequence.

Isolated: The term “isolated” or “isolation” as used herein means that a material has been removed by the hand of man and exists apart from its original, native environment and is therefore not a product of nature. An isolated material or molecule (such as a DNA molecule or enzyme) may exist in a purified form or may exist in a non-native environment such as, for example, in a transgenic host cell. For example, a naturally occurring polynucleotide or polypeptide present in a living plant is not isolated, but the same polynucleotide or polypeptide, separated from some or all of the coexisting materials in the natural system, is isolated. Such polynucleotides can be part of a vector and/or such polynucleotides or polypeptides could be part of a composition, and would be isolated in that such a vector or composition is not part of its original environment. Preferably, the term “isolated” when used in relation to a nucleic acid molecule, as in “an isolated nucleic acid sequence” refers to a nucleic acid sequence that is identified and separated from at least one contaminant nucleic acid molecule with which it is ordinarily associated in its natural source. Isolated nucleic acid molecule is nucleic acid molecule present in a form or setting that is different from that in which it is found in nature. In contrast, non-isolated nucleic acid molecules are nucleic acid molecules such as DNA and RNA, which are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs, which encode a multitude of proteins. However, an isolated nucleic acid sequence comprising for example SEQ ID NO: 1 includes, by way of example, such nucleic acid sequences in cells which ordinarily contain SEQ ID NO: 1 where the nucleic acid sequence is in a chromosomal or extrachromosomal location different from that of natural cells, or is otherwise flanked by a different nucleic acid sequence than that found in nature. The isolated nucleic acid sequence may be present in single-stranded or double-stranded form. When an isolated nucleic acid sequence is to be utilized to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded).

Minimal Promoter: the term “minimal promoter” is interchangeable with the terms “basal promoter” or “core promoter”. The minimal promoter comprises promoter elements, particularly a TATA element, and is inactive or has greatly reduced promoter activity in the absence of upstream activation. The minimal promoter plays a central role in regulating initiation of transcription. Specific DNA sequences within the minimal promoter bind the factors, for example transcription factors, that allow the assembly of a functional preinitiation complex. In the presence of a suitable transcription factor, the minimal promoter functions to permit transcription. Detailed definition of minimal promoters may be found in Smale and Kadonaga (2003, Annual review of Biochemistry 77, pages 449-479), Yamamoto et al (2007, BMC Genomics 8 (67), Yamamoto et al (2007, Nucleic Acid Research 35(18) pages 6219-6226). In a preferred embodiment, the minimal promoter is a polymerase II minimal promoter.

NEENA: see “Nucleic acid expression enhancing nucleic acid”.

Non-coding: The term “non-coding” refers to sequences of nucleic acid molecules that do not encode part or all of an expressed protein. Non-coding sequences include but are not limited to introns, enhancers, promoter regions, 3′ untranslated regions, and 5′ untranslated regions.

Nucleic acid expression enhancing nucleic acid (NEENA): The term “nucleic acid expression enhancing nucleic acid” refers to a sequence and/or a nucleic acid molecule of a specific sequence having the intrinsic property to enhance expression of a nucleic acid under the control of a promoter to which the NEENA is functionally linked. Unlike promoter sequences, the NEENA as such is not able to drive expression. In order to fulfill the function of enhancing expression of a nucleic acid molecule functionally linked to the NEENA, the NEENA itself has to be functionally linked to a promoter. In distinction to enhancer sequences known in the art, the NEENA is acting in cis but not in trans and has to be located close to the transcription start site of the nucleic acid to be expressed.

Nucleic acids and nucleotides: The terms “Nucleic Acids” and “Nucleotides” refer to naturally occurring or synthetic or artificial nucleic acid or nucleotides. The terms “nucleic acids” and “nucleotides” comprise deoxyribonucleotides or ribonucleotides or any nucleotide analogue and polymers or hybrids thereof in either single- or double-stranded, sense or antisense form. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used interchangeably herein with “gene”, “cDNA, “mRNA”, “oligonucleotide,” and “polynucleotide”. Nucleotide analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, halo, SH, SR, NH2, NHR, NR2, or CN. Short hairpin RNAs (shRNAs) also can comprise non-natural elements such as non-natural bases, e.g., ionosin and xanthine, non-natural sugars, e.g., 2′-methoxy ribose, or non-natural phosphodiester linkages, e.g., methylphosphonates, phosphorothioates and peptides.

Nucleic acid sequence: The phrase “nucleic acid sequence” refers to a single or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length. A “target region” of a nucleic acid is a portion of a nucleic acid that is identified to be of interest. A “coding region” of a nucleic acid is the portion of the nucleic acid, which is transcribed and translated in a sequence-specific manner to produce into a particular polypeptide or protein when placed under the control of appropriate regulatory sequences. The coding region is said to encode such a polypeptide or protein.

Oligonucleotide: The term “oligonucleotide” refers to an oligomer or polymer of ribonucleic acid (RNA) or deoxyribonucleic acid (DNA) or mimetics thereof, as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. An oligonucleotide preferably includes two or more nucleomonomers covalently coupled to each other by linkages (e.g., phosphodiesters) or substitute linkages.

Overhang: An “overhang” is a relatively short single-stranded nucleotide sequence on the 5′- or 3′-hydroxyl end of a double-stranded oligonucleotide molecule (also referred to as an “extension,” “protruding end,” or “sticky end”).

Plant: is generally understood as meaning any eukaryotic single- or multi-celled organism or a cell, tissue, organ, part or propagation material (such as seeds or fruit) of same which is capable of photosynthesis. Included for the purpose of the invention are all genera and species of higher and lower plants of the Plant Kingdom. Annual, perennial, monocotyledonous and dicotyledonous plants are preferred. The term includes the mature plants, seed, shoots and seedlings and their derived parts, propagation material (such as seeds or microspores), plant organs, tissue, protoplasts, callus and other cultures, for example cell cultures, and any other type of plant cell grouping to give functional or structural units. Mature plants refer to plants at any desired developmental stage beyond that of the seedling. Seedling refers to a young immature plant at an early developmental stage. Annual, biennial, monocotyledonous and dicotyledonous plants are preferred host organisms for the generation of transgenic plants. The expression of genes is furthermore advantageous in all ornamental plants, useful or ornamental trees, flowers, cut flowers, shrubs or lawns. Plants which may be mentioned by way of example but not by limitation are angiosperms, bryophytes such as, for example, Hepaticae (liverworts) and Musci (mosses); Pteridophytes such as ferns, horsetail and club mosses; gymnosperms such as conifers, cycads, ginkgo and Gnetatae; algae such as Chlorophyceae, Phaeophpyceae, Rhodophyceae, Myxophyceae, Xanthophyceae, Bacillariophyceae (diatoms), and Euglenophyceae. Preferred are plants which are used for food or feed purpose such as the families of the Leguminosae such as pea, alfalfa and soya; Gramineae such as rice, especially Oryza sativa, maize, especially Zea mays, wheat, barley, sorghum, millet, rye, triticale, or oats; the family of the Umbelliferae, especially the genus Daucus, very especially the species carota (carrot) and Apium, very especially the species Graveolens dulce (celery) and many others; the family of the Solanaceae, especially the genus Lycopersicon, very especially the species esculentum (tomato) and the genus Solanum, very especially the species tuberosum (potato) and melongena (egg plant), and many others (such as tobacco); and the genus Capsicum, very especially the species annuum (peppers) and many others; the family of the Leguminosae, especially the genus Glycine, very especially the species max (soybean), alfalfa, pea, lucerne, beans or peanut and many others; and the family of the Cruciferae (Brassicacae), especially the genus Brassica, very especially the species napus (oil seed rape), campestris (beet), oleracea cv Tastie (cabbage), oleracea cv Snowball Y (cauliflower) and oleracea cv Emperor (broccoli); and of the genus Arabidopsis, very especially the species thaliana and many others; the family of the Compositae, especially the genus Lactuca, very especially the species sativa (lettuce) and many others; the family of the Asteraceae such as sunflower, Tagetes, lettuce or Calendula and many other; the family of the Cucurbitaceae such as melon, pumpkin/squash or zucchini, and linseed. Further preferred are cotton, sugar cane, hemp, flax, chillies, and the various tree, nut and wine species.

Polypeptide: The terms “polypeptide”, “peptide”, “oligopeptide”, “polypeptide”, “gene product”, “expression product” and “protein” are used interchangeably herein to refer to a polymer or oligomer of consecutive amino acid residues.

Pre-protein: Protein, which is normally targeted to a cellular organelle, such as a chloroplast, and still comprising its transit peptide.

Primary transcript: The term “primary transcript” as used herein refers to a premature RNA transcript of a gene. A “primary transcript” for example still comprises introns and/or is not yet comprising a polyA tail or a cap structure and/or is missing other modifications necessary for its correct function as transcript such as for example trimming or editing.

Promoter: The terms “promoter”, or “promoter sequence” are equivalents and as used herein, refer to a DNA sequence which when ligated to a nucleotide sequence of interest is capable of controlling the transcription of the nucleotide sequence of interest into RNA. Such promoters can for example be found in the following public databases http://www.grassius.org/grasspromdb.html, http://mendel.cs.rhul.ac.uk/mendel.php?topic=plantprom, http://ppdb.gene.nagoya-u.ac.jp/cgibin/index.cgi. Promoters listed there may be addressed with the methods of the invention and are herewith included by reference. A promoter is located 5′ (i.e., upstream), proximal to the transcriptional start site of a nucleotide sequence of interest whose transcription into mRNA it controls, and provides a site for specific binding by RNA polymerase and other transcription factors for initiation of transcription. Said promoter comprises for example the at least 10 kb, for example 5 kb or 2 kb proximal to the transcription start site. It may also comprise the at least 1500 bp proximal to the transcriptional start site, preferably the at least 1000 bp, more preferably the at least 500 bp, even more preferably the at least 400 bp, the at least 300 bp, the at least 200 bp or the at least 100 bp. In a further preferred embodiment, the promoter comprises the at least 50 bp proximal to the transcription start site, for example, at least 25 bp. The promoter does not comprise exon and/or intron regions or 5′ untranslated regions. The promoter may for example be heterologous or homologous to the respective plant. A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety). Suitable promoters can be derived from genes of the host cells where expression should occur or from pathogens for this host cells (e.g., plants or plant pathogens like plant viruses). A plant specific promoter is a promoter suitable for regulating expression in a plant. It may be derived from a plant but also from plant pathogens or it might be a synthetic promoter designed by man. If a promoter is an inducible promoter, then the rate of transcription increases in response to an inducing agent. Also, the promoter may be regulated in a tissue-specific or tissue preferred manner such that it is only or predominantly active in transcribing the associated coding region in a specific tissue type(s) such as leaves, roots or meristem. The term “tissue specific” as it applies to a promoter refers to a promoter that is capable of directing selective expression of a nucleotide sequence of interest to a specific type of tissue (e.g., petals) in the relative absence of expression of the same nucleotide sequence of interest in a different type of tissue (e.g., roots). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected. The term “cell type specific” as applied to a promoter refers to a promoter, which is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., GUS activity staining, GFP protein or immunohistochemical staining. The term “constitutive” when made in reference to a promoter or the expression derived from a promoter means that the promoter is capable of directing transcription of an operably linked nucleic acid molecule in the absence of a stimulus (e.g., heat shock, chemicals, light, etc.) in the majority of plant tissues and cells throughout substantially the entire lifespan of a plant or part of a plant. Typically, constitutive promoters are capable of directing expression of a transgene in substantially any cell and any tissue.

Promoter specificity: The term “specificity” when referring to a promoter means the pattern of expression conferred by the respective promoter. The specificity describes the tissues and/or developmental status of a plant or part thereof, in which the promoter is conferring expression of the nucleic acid molecule under the control of the respective promoter. Specificity of a promoter may also comprise the environmental conditions, under which the promoter may be activated or down-regulated such as induction or repression by biological or environmental stresses such as cold, drought, wounding or infection.

Purified: As used herein, the term “purified” refers to molecules, either nucleic or amino acid sequences that are removed from their natural environment, isolated or separated. “Substantially purified” molecules are at least 60% free, preferably at least 75% free, and more preferably at least 90% free from other components with which they are naturally associated. A purified nucleic acid sequence may be an isolated nucleic acid sequence.

Recombinant: The term “recombinant” with respect to nucleic acid molecules refers to nucleic acid molecules produced by recombinant DNA techniques. Recombinant nucleic acid molecules may also comprise molecules, which as such does not exist in nature but are modified, changed, mutated or otherwise manipulated by man. Preferably, a “recombinant nucleic acid molecule” is a non-naturally occurring nucleic acid molecule that differs in sequence from a naturally occurring nucleic acid molecule by at least one nucleic acid. A “recombinant nucleic acid molecule” may also comprise a “recombinant construct” which comprises, preferably operably linked, a sequence of nucleic acid molecules not naturally occurring in that order. Preferred methods for producing said recombinant nucleic acid molecule may comprise cloning techniques, directed or non-directed mutagenesis, synthesis or recombination techniques.

“Seed-specific promoter” in the context of this invention means a promoter which is regulating transcription of a nucleic acid molecule under control of the respective promoter in seeds wherein the transcription in any tissue or cell of the seeds contribute to more than 90%, preferably more than 95%, more preferably more than 99% of the entire quantity of the RNA transcribed from said nucleic acid sequence in the entire plant during any of its developmental stage. The term “seed-specific expression” is to be understood accordingly.

“Seed-preferential promoter” in the context of this invention means a promoter which is regulating transcription of a nucleic acid molecule under control of the respective promoter in seeds wherein the transcription in any tissue or cell of the seeds contribute to more than 50%, preferably more than 70%, more preferably more than 80% of the entire quantity of the RNA transcribed from said nucleic acid sequence in the entire plant during any of its developmental stage. The term “seed-preferential expression” is to be understood accordingly.

Sense: The term “sense” is understood to mean a nucleic acid molecule having a sequence which is complementary or identical to a target sequence, for example a sequence which binds to a protein transcription factor and which is involved in the expression of a given gene. According to a preferred embodiment, the nucleic acid molecule comprises a gene of interest and elements allowing the expression of the said gene of interest.

SIE: SIE or stress-induction element is a sequence motif that is a transcription factor or regulatory RNA interaction or binding sequence in a promoter that is mediating induction of gene expression derived from the promoter to which it is functionally linked.

Significant increase or decrease: An increase or decrease, for example in enzymatic activity or in gene expression, that is larger than the margin of error inherent in the measurement technique, preferably an increase or decrease by about 2-fold or greater of the activity of the control enzyme or expression in the control cell, more preferably an increase or decrease by about 5-fold or greater, and most preferably an increase or decrease by about 10-fold or greater.

Small nucleic acid molecules: “small nucleic acid molecules” are understood as molecules consisting of nucleic acids or derivatives thereof such as RNA or DNA. They may be double-stranded or single-stranded and are between about 15 and about 30 bp, for example between 15 and 30 bp, more preferred between about 19 and about 26 bp, for example between 19 and 26 bp, even more preferred between about 20 and about 25 bp for example between 20 and 25 bp. In a especially preferred embodiment the oligonucleotides are between about 21 and about 24 bp, for example between 21 and 24 bp. In a most preferred embodiment, the small nucleic acid molecules are about 21 bp and about 24 bp, for example 21 bp and 24 bp.

Substantially complementary: In its broadest sense, the term “substantially complementary”, when used herein with respect to a nucleotide sequence in relation to a reference or target nucleotide sequence, means a nucleotide sequence having a percentage of identity between the substantially complementary nucleotide sequence and the exact complementary sequence of said reference or target nucleotide sequence of at least 60%, more desirably at least 70%, more desirably at least 80% or 85%, preferably at least 90%, more preferably at least 93%, still more preferably at least 95% or 96%, yet still more preferably at least 97% or 98%, yet still more preferably at least 99% or most preferably 100% (the later being equivalent to the term “identical” in this context). Preferably identity is assessed over a length of at least 19 nucleotides, preferably at least 50 nucleotides, more preferably the entire length of the nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J Mol. Biol. 48: 443-453; as defined above). A nucleotide sequence “substantially complementary” to a reference nucleotide sequence hybridizes to the reference nucleotide sequence under low stringency conditions, preferably medium stringency conditions, most preferably high stringency conditions (as defined above).

Terminator: The term “terminator” “transcription terminator” or “transcription terminator sequence” as used herein is intended to mean a sequence located in the 3″UTR of a gene that causes a polymerase to stop forming phosphodiester bonds and release the nascent transcript. As used herein, the terminator comprises the entire 3″UTR structure necessary for efficient production of a messenger RNA. The terminator sequence leads to or initiates a stop of transcription of a nucleic acid sequence initiated from a promoter. Preferably, a transcription terminator sequence furthermore comprises sequences which cause polyadenylation of the transcript. A transcription terminator may, for example, comprise one or more polyadenylation signal sequences, one or more polyadenylation attachment sequences, and downstream sequence of various lengths which causes termination of transcription. It has to be understood that also sequences downstream of sequences coding for the 3″-untranslated region of an expressed RNA transcript may be part of a transcription terminator although the sequence itself is not expressed as part of the RNA transcript. Furthermore, a transcription terminator may comprise additional sequences, which may influence its functionality, such a 3″-untranslated sequences (ie. sequences of a gene following the stop-codon of the coding sequence). Transcription termination may involve various mechanisms including but not limited to induced dissociation of RNA polymerase II from their DNA template.

Transgene: The term “transgene” as used herein refers to any nucleic acid sequence, which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence.

Transgenic: The term transgenic when referring to an organism means transformed, preferably stably transformed, with a recombinant DNA molecule that preferably comprises a suitable promoter operatively linked to a DNA sequence of interest.

Vector: As used herein, the term “vector” refers to a nucleic acid molecule capable of transporting another nucleic acid molecule to which it has been linked. One type of vector is a genomic integrated vector, or “integrated vector”, which can become integrated into the chromosomal DNA of the host cell. Another type of vector is an episomal vector, i.e., a nucleic acid molecule capable of extra-chromosomal replication. Vectors capable of directing the expression of genes to which they are operatively linked are referred to herein as “expression vectors”. In the present specification, “plasmid” and “vector” are used interchangeably unless otherwise clear from the context. Expression vectors designed to produce RNAs as described herein in vitro or in vivo may contain sequences recognized by any RNA polymerase, including mitochondrial RNA polymerase, RNA pol I, RNA pol II, and RNA pol III. These vectors can be used to transcribe the desired RNA molecule in the cell according to this invention. A plant transformation vector is to be understood as a vector suitable in the process of plant transformation.

Wild-type: The term “wild-type”, “natural” or “natural origin” means with respect to an organism, polypeptide, or nucleic acid sequence, that said organism is naturally occurring or available in at least one naturally occurring organism which is not changed, mutated, or otherwise manipulated by man.

### EXAMPLES

### Chemicals and Common Methods

Unless indicated otherwise, cloning procedures carried out for the purposes of the present invention including restriction digest, agarose gel electrophoresis, purification of nucleic acids, ligation of nucleic acids, transformation, selection and cultivation of bacterial cells were performed as described (Sambrook et al., 1989). Sequence analyses of recombinant DNA were performed with a laser fluorescence DNA sequencer (Applied Biosystems, Foster City, Calif., USA) using the Sanger technology (Sanger et al., 1977). Unless described otherwise, chemicals and reagents were obtained from Sigma Aldrich (Sigma Aldrich, St. Louis, USA), from Promega (Madison, Wis., USA), Duchefa (Haarlem, The Netherlands) or Invitrogen (Carlsbad, Calif., USA). Restriction endonucleases were from New England Biolabs (Ipswich, Mass., USA) or Roche Diagnostics GmbH (Penzberg, Germany). Oligonucleotides were synthesized by Eurofins MWG Operon (Ebersberg, Germany).

### Example 1

### Cloning of the Promoter Sequences

The nucleic acid sequences of the promoters (SEQ ID NOs: 4, 9, 14, 19, 24, 29, 34, 39, 41, 42, and 43) were chemically synthesized through a commercial provider. Each promoter sequence was flanked from both ends immediately with an 8-mer sequence of ATTTAAAT that was the recognition sequence of restriction enzyme Swal. The promoter sequences were then subcloned as Swal fragments into a parental vector at Swal site by a conventional method. The resulted constructs are destination vectors, according to the Gateway terminology of Invitrogen (Lifetech). These destination vectors contained as functional elements within the T-DNA borders: a plant selectable marker; a screenable marker expression cassette; and a Gateway cassette that located downstream of the said promoters. The Gateway cassette was intended for LR in vivo recombination with the nucleic acid sequence of interest already cloned in an entry clone.

To test the activity of the promoters, an Escherichia coli Beta-glucuronidase (GUS) coding sequence that resides in the Gateway cassette of an entry clone was introduced to each of the destination vectors via an LR in vivo recombination. The descendent constructs, which are expression vectors (SEQ ID NO: 5, 10, 15, 20, 25, 30, 35 and 40), were individually transformed into Agrobacterium strain LBA4044 according to methods well known in the art. The agrobacterium strains were used for Oryza sativa transformation.

### Example 2

### Cloning of the Promoter Sequences with Intron

An intron sequence (SEQ ID NO: 44) is attached to the sequences of the promoters with SEQ ID NOs: 4, 9, 14, 19, 24, 29, 34, 39, 41, 42, and 43. The resulting expression regulating nucleotide sequences (SEQ ID NOs: 45, 46, 47, 48, 49, 50, 51, 52, 53, 54, and 55) are then chemically synthesized through a commercial provider. Each expression regulating nucleotide sequence is flanked from both ends immediately with an 8-mer sequence of ATTTAAAT that is the recognition sequence of restriction enzyme Swal. The expression regulating nucleotide sequences are then sub-cloned as Swal fragments into a parental vector at Swal site by a conventional method. The resulted constructs are destination vectors, according to the Gateway terminology of Invitrogen (Lifetech). These destination vectors containes as functional elements within the T-DNA borders: a plant selectable marker; a screenable marker expression cassette; and a Gateway cassette that located downstream of the said promoters. The Gateway cassette is used for LR in vivo recombination with the nucleic acid sequence of interest already cloned in an entry clone.

To test the activity of the expression regulating nucleotide sequences, an Escherichia coli Betaglucuronidase (GUS) coding sequence that resides in the Gateway cassette of an entry clone is introduced to each of the destination vectors via an LR in vivo recombination. The descendent constructs, which are expression vectors, are individually transformed into Agrobacterium strain LBA4044 according to methods well known in the art. The agrobacterium strains are used for Oryza sativa transformation.

### Example 3

### Generation of Transgenic Rice Plants

Agrobacterium cells containing the respective expression vectors of Example 1 were used to transform Oryza sativa plants. Mature dry seeds of the rice japonica cultivar Nipponbare were dehusked. Sterilization was carried out by incubating for one minute in 70% ethanol, followed by 30 minutes in 0.2% HgCl₂, followed by a 6 times 15 minutes wash with sterile distilled water. The sterile seeds were then germinated on a medium containing 2.4-D (callus induction medium). After incubation in the dark for four weeks, embryogenic, scutellum-derived calli were excised and propagated on the same medium. After two weeks, the calli were multiplied or propagated by subculture on the same medium for another 2 weeks. Embryogenic callus pieces were sub-cultured on fresh medium 3 days before co-cultivation (to boost cell division activity).

Agrobacterium strain LBA4404 containing the respective expression vector was used for co-cultivation. Agrobacterium was inoculated on AB medium with the appropriate antibiotics and cultured for 3 days at 28° C. The bacteria were then collected and suspended in liquid cocultivation medium to a density (OD₆₀₀) of about 1. The suspension was then transferred to a Petri dish and the calli immersed in the suspension for 15 minutes. The callus tissues were then blotted dry on a filter paper and transferred to solidified, co-cultivation medium and incubated for 3 days in the dark at 25° C. Co-cultivated calli were grown on 2.4-D-containing medium for 4 weeks in the dark at 28° C. in the presence of a selection agent. During this period, rapidly growing resistant callus islands developed. After transfer of this material to a regeneration medium and incubation in the light, the embryogenic potential was released and shoots developed in the next four to five weeks. Shoots were excised from the calli and incubated for 2 to 3 weeks on an auxin-containing medium from which they were transferred to soil. Hardened shoots were grown under high humidity and short days in a greenhouse.

Approximately 35 independent T0 rice transformants were generated for each construct. The primary transformants were transferred from a tissue culture chamber to a greenhouse. After a quantitative PCR analysis to verify copy number of the T-DNA insert, only single copy transgenic plants that exhibited tolerance to the selection agent were kept for harvest of T1 seed. Seeds were then harvested three to five months after transplanting. The method yielded single locus transformants at a rate of over 50% (Aldemita and Hodges, 1996, Chan et al., 1993, Hiei et al., 1994).

The expression constructs of Example 2 are transformed into rice accordingly.

### Example 4

### Analysis of the Basal Activity of Promoters SEQ ID NOs: 4, 9, 14, 19, 24, 29, 34, and 39

The basal activity of the promoters (SEQ ID NOs: 4, 9, 14, 19, 24, 29, 34, and 39) in transgenic rice was tested by detecting the GUS expression level in callus, regenerated plantlets, and samples from the T0 plants growing in the greenhouse.

Twelve to twenty pieces of callus and about 12 regenerated plantlets transformed with any of the promoters with SEQ ID NOs: 4, 9, 14, 19, 24, 29, 34 and 39 were sampled during rice transformation, and subjected for GUS staining analysis according to Jefferson et al., 1987.

Leaf samples of seedlings and adult plants, young inflorescence, and mature seed were collected from 20 to 25 single copy T0 plants per construct. Each sample was ground to powder at −80 degree Celsius and was then extracted with 50 mM phosphate buffer (pH7.0). The extracts were tested for their GUS activity via a kinetic fluorimetrical assay by using 4-methylumbelliferyl-beta-D-glucuronide (MUG) as substrate (Jefferson et al. 1987). The GUS activity in mature seeds was also examined by GUS staining. The results showed no or hardly detectable basal GUS expression of the tested promoters under unstressed conditions (see Table 1).

TABLE 1
Analysis of GUS Expression in Tissues of unstressed plants

| Promoter | GUS activity in plant tissue | | | | | | | |
|---|---|---|---|---|---|---|---|---|
| SEQ ID | leaf | Leaf | | | GUS staining in seed | | | |
| NO | seedling | Adult | inflorescence | Seeds | embryo | emdosperm | aleurone | husk |
| 4 | none | none | None | none | none | none | none | none |
| 9 | none | none | None | none | none | none | none | none |
| 14 | none | none | None | none | none | none | none | none |
| 19 | none | none | Detectable | none | none | none | none | none |
| 24 | none | none | None | none | none | none | none | none |
| 29 | detectable | none | None | none | none | none | none | none |
| 34 | none | none | None | none | none | none | none | none |
| 39 | detectable | none | None | none | none | none | none | none |

### Example 5

### Analysis of the Basal Activity of Promoters with Intron SEQ ID NOs: 48, 49, 50, 51, 52, 53, 54, and 55

The basal activity of the promoters (SEQ ID NOs: 48, 49, 50, 51, 52, 53, 54, and 55) is tested by detecting the GUS expression level in callus, regenerated plantlets, and samples from the T0 plants growing in the greenhouse.

Twelve to twenty pieces of callus and about 12 regenerated plantlets transformed with any of the expression regulating nucleotide sequences comprising the promoters of SEQ ID NOs: 48, 49, 50, 51, 52, 53, 54, and 55 are sampled during rice transformation, and subjected for GUS staining analysis according to Jefferson et al., 1987.

Leaf samples of seedlings and adult plants, young inflorescence, and mature seed are collected from 20 to 25 single copy T0 plants per construct. Each sample is ground to powder at −80 degree Celsius and is then extracted with 50 mM phosphate buffer (pH7.0). The extracts are tested for their GUS activity via a kinetic fluorimetrical assay by using 4-methylumbelliferyl-beta-D-glucuronide (MUG) as substrate (Jefferson et al. 1987). The GUS activity in mature seeds is also examined by GUS staining. The results show no or hardly detectable basal GUS expression of the tested promoters under unstressed conditions.

### Example 6

### Analysis of Promoter Activity in Response to Drought Treatment SEQ ID NOs: 4, 9, 14, 19, 24, 29, 34, and 39

The response of promoters (SEQ ID NOs: 4, 9, 14, 19, 24, 29, 34, and 39) to drought treatment was examined in T1 transgenic rice. T1 seeds from 6 single-copy T0 events per promoter were checked for the expression of GFP, a visual marker present in the construct, and transgenic seeds were selected and sown in soil. Nine transgenic plants per event were grown in drought stressed regime. The same amount of plants from these events was grown under unstressed conditions as control. Leaf samples at age of 3 w, 6 w, and 9 w, as well as along the period of drought treatment were collected for quantitative analysis of GUS activity. Six leaf samples per plant were selected resulting in a total of 54 samples per investigated time point. The drought treatment included two applications of drought stress (DS). The first drought stress was applied in the seedling stage: begin of the first droght stress (DS symptom) and after the end of the first droght stress cycle (DS recovery). The second drought stress was applied in the reproductive stage: begin (Late DS start) and the end (Late DS end) of second drought stress.

Each tested promoter showed a distinct drought stress induced GUS expression pattern in all tissues tested compared to unstressed transgenic plants of the same line.

TABLE 2
Fold change of GUS Expression of unstressed vs. drought stressed plants
in leafs. The fold change was calculated by dividing the mean of the GUS
expression under drought stress by the mean GUS expression under
unstressed conditions (mean DS/mean unstressed, DS = Drought Stress).

| Promoter | DS | DS | Late | Late |
|---|---|---|---|---|
| SEQ ID NO | symptom | recovery | DS start | DS end |
| 4 | 2.0 | 1.1 | 1.7 | 0.9 |
| 9 | 1.3 | 1.1 | 1.3 | 1.0 |
| 14 | 1.6 | 1.4 | 1.6 | 1.2 |
| 19 | 1.6 | 1.2 | 1.7 | 2.2 |
| 24 | 1.9 | 1.5 | 1.3 | 0.8 |
| 29 | 1.1 | 1.8 | 2.3 | 1.1 |
| 34 | 1.6 | 1.5 | 2.4 | 0.7 |
| 39 | 1.3 | 1.4 | 1.7 | 2.0 |

The promoter with SEQ ID NO 4 showed two expression peaks at the beginning of each drought stress application. The promoter with SEQ ID NO 9 showed increased GUS expression at the beginning of the first and second drought stress application representing an early drought response. The promoter with SEQ ID NO 14 showed also an expression peak at the beginning of the drought treatments, but stayed on a higher level after the drought stress was over. The promoter with SEQ ID NO 14 allows expressing genes specifically during and after a drought stress event enabling a long term response to the drought stress. The promoter with SEQ ID NO 19 shows a similar expression pattern than the promoter with SEQ ID NO 14 except that the promoter with SEQ ID NO 19 showed the highest expression after the second drought stress application. The promoter with SEQ ID NO 24 showed an expression peak at the beginning of the first drought treatment with a following decreasing expression over time reaching the non-increased expression level at the last measured time point. The promoter with SEQ ID NO 29 showed an increased expression level after the first drought treatment and the beginning of the second drought treatment. The promoter with SEQ ID NO 34 showed an increased GUS expression over the two drought treatments reaching a maximum at the beginning of the second drought treatment. The promoter with SEQ ID NO 39 showed a steadily increasing GUS expression over the measured time points.

In summary, the set of identified drought stress inducible promoters are valuable tools towards a fine-tuned optimized expression of trans-genes.

### Example 7

### Analysis of the Activity of Combinations of Promoter with Intron in Response to Drought Treatment SEQ ID NOs: 48, 49, 50, 51, 52, 53, 54, and 55

The response of promoters (SEQ ID NOs: 48, 49, 50, 51, 52, 53, 54, and 55) to drought treatment is examined in T1 transgenic rice. T1 seeds from 6 single-copy T0 events per promoter are checked for the expression of GFP, a visual marker present in the construct, and transgenic seeds are selected and sown in soil. Nine transgenic plants per event are grown in drought stressed regime. The same amount of plants from these events is grown under unstressed conditions as one control. Leaf samples at age of 3 w, 6 w, and 9 w, as well as along the period of drought treatment are collected for quantitative analysis of GUS activity. Six samples per plant are selected resulting in a total of 54 samples per investigated time point. The drought treatment included two applications of drought stress (DS). The first drought stress is applied in the seedling stage: begin of the first drought stress (DS symptom) and after the end of the first drought stress cycle (DS recovery). The second drought stress is applied in the reproductive stage: begin (Late DS start) and the end (Late DS end) of second drought stress. Inflorescence sample and mature seed from plants with and without drought treatment are also tested for GUS expression.

The results show that the GUS expression is induced in by drought stress compared to the control.

### Example 8

### Analysis of the Basal Activity of Promoter SEQ ID NO: 41

The basal activity of the promoter (SEQ ID NO: 41) in transgenic rice is tested by detecting the GUS expression level in callus, regenerated plantlets, and samples from the T0 plants growing in the greenhouse.

Twelve to twenty pieces of callus and about 12 regenerated plantlets transformed with any of the promoter with SEQ ID NO: 41 are sampled during rice transformation, and subjected for GUS staining analysis according to Jefferson et al., 1987.

Leaf samples of seedlings and adult plants, young inflorescence, and mature seed are collected from 20 to 25 single copy T0 plants per construct. Each sample is ground to powder at −80 degree Celsius and was then extracted with 50 mM phosphate buffer (pH7.0). The extracts are tested for their GUS activity via a kinetic fluorimetrical assay by using 4-methylumbelliferyl-beta-D-glucuronide (MUG) as substrate (Jefferson et al. 1987). The GUS activity in mature seeds is also examined by GUS staining. The results show no or hardly detectable basal GUS expression of the tested promoter (SEQ ID NO: 41) under unstressed conditions.

### Example 9

### Analysis of the Basal Activity of Promoter SEQ ID NO: 45

The basal activity of the promoter (SEQ ID NO: 45) in transgenic rice is tested by detecting the GUS expression level in callus, regenerated plantlets, and samples from the T0 plants growing in the greenhouse.

Twelve to twenty pieces of callus and about 12 regenerated plantlets transformed with any of the promoter with SEQ ID NO: 45 are sampled during rice transformation, and subjected for GUS staining analysis according to Jefferson et al., 1987.

Leaf samples of seedlings and adult plants, young inflorescence, and mature seed are collected from 20 to 25 single copy T0 plants per construct. Each sample is ground to powder at −80 degree Celsius and was then extracted with 50 mM phosphate buffer (pH7.0). The extracts are tested for their GUS activity via a kinetic fluorimetrical assay by using 4-methylumbelliferyl-beta-D-glucuronide (MUG) as substrate (Jefferson et al. 1987). The GUS activity in mature seeds is also examined by GUS staining. The results show no or hardly detectable basal GUS expression of the tested promoter (SEQ ID NO: 45) under unstressed conditions.

### Example 10

### Analysis of Promoter Activity in Response to Drought Treatment of Promoter SEQ ID NO: 41

The response of promoter with SEQ ID NO: 41 to drought treatment is examined in T1 transgenic rice. T1 seeds from 6 single-copy T0 events are checked for the expression of GFP, a visual marker present in the construct, and transgenic seeds are selected and sown in soil. Nine transgenic plants per event are grown in drought stressed regime. The same amount of plants from these events is grown under unstressed conditions as one control. Leaf samples at age of 3 w, 6 w, and 9 w, as well as along the period of drought treatment are collected for quantitative analysis of GUS activity. Inflorescence sample and mature seed from plants with and without drought treatment are also tested for GUS expression.

The results show, that the GUS expression is induced by drought stress compared to the control.

### Example 11

### Analysis of Promoter with Intron Activity in Response to Drought Treatment of Promoter SEQ ID NO: 45

The response of promoter with SEQ ID NO: 45 to drought treatment is examined in T1 transgenic rice. T1 seeds from 6 single-copy T0 events are checked for the expression of GFP, a visual marker present in the construct, and transgenic seeds are selected and sown in soil. Nine transgenic plants per event are grown in drought stressed regime. The same amount of plants from these events is grown under unstressed conditions as one control. Leaf samples at age of 3 w, 6 w, and 9 w, as well as along the period of drought treatment are collected for quantitative analysis of GUS activity. Inflorescence sample and mature seed from plants with and without drought treatment are also tested for GUS expression.

The results show, that the GUS expression is induced by drought stress compared to the control.

### Example 12

### Analysis of the Basal Activity of Promoter SEQ ID NO: 42

The basal activity of the promoter (SEQ ID NO: 42) in transgenic rice is tested by detecting the GUS expression level in callus, regenerated plantlets, and samples from the T0 plants growing in the greenhouse.

Twelve to twenty pieces of callus and about 12 regenerated plantlets transformed with any of the promoter with SEQ ID NO: 42 are sampled during rice transformation, and subjected for GUS staining analysis according to Jefferson et al., 1987.

Leaf samples of seedlings and adult plants, young inflorescence, and mature seed are collected from 20 to 25 single copy T0 plants per construct. Each sample is ground to powder at −80 degree Celsius and was then extracted with 50 mM phosphate buffer (pH7.0). The extracts are tested for their GUS activity via a kinetic fluorimetrical assay by using 4-methylumbelliferyl-beta-D-glucuronide (MUG) as substrate (Jefferson et al. 1987). The GUS activity in mature seeds is also examined by GUS staining. The results show no or hardly detectable basal GUS expression of the tested promoter (SEQ ID NO: 42) under unstressed conditions.

### Example 13

### Analysis of the Basal Activity of Promoter SEQ ID NO: 46

The basal activity of the promoter (SEQ ID NO: 46) in transgenic rice is tested by detecting the GUS expression level in callus, regenerated plantlets, and samples from the T0 plants growing in the greenhouse.

Twelve to twenty pieces of callus and about 12 regenerated plantlets transformed with any of the promoter with SEQ ID NO: 46 are sampled during rice transformation, and subjected for GUS staining analysis according to Jefferson et al., 1987.

Leaf samples of seedlings and adult plants, young inflorescence, and mature seed are collected from 20 to 25 single copy T0 plants per construct. Each sample is ground to powder at −80 degree Celsius and was then extracted with 50 mM phosphate buffer (pH7.0). The extracts are tested for their GUS activity via a kinetic fluorimetrical assay by using 4-methylumbelliferyl-beta-D-glucuronide (MUG) as substrate (Jefferson et al. 1987). The GUS activity in mature seeds is also examined by GUS staining. The results show no or hardly detectable basal GUS expression of the tested promoter (SEQ ID NO: 46) under unstressed conditions.

### Example 14

### Analysis of Promoter Activity in Response to Drought Treatment of Promoter SEQ ID NO: 42

The response of promoter with SEQ ID NO: 42 to drought treatment is examined in T1 transgenic rice. T1 seeds from 6 single-copy T0 events are checked for the expression of GFP, a visual marker present in the construct, and transgenic seeds are selected and sown in soil. Nine transgenic plants per event are grown in drought stressed regime. The same amount of plants from these events is grown under unstressed conditions as one control. Leaf samples at age of 3 w, 6 w, and 9 w, as well as along the period of drought treatment are collected for quantitative analysis of GUS activity. Inflorescence sample and mature seed from plants with and without drought treatment are also tested for GUS expression.

The results show, that the GUS expression is induced by drought stress compared to the control.

### Example 15

### Analysis of Promoter with Intron Activity in Response to Drought Treatment of Promoter SEQ ID NO: 46

The response of promoter with SEQ ID NO: 46 to drought treatment is examined in T1 transgenic rice. T1 seeds from 6 single-copy T0 events are checked for the expression of GFP, a visual marker present in the construct, and transgenic seeds are selected and sown in soil. Nine transgenic plants per event are grown in drought stressed regime. The same amount of plants from these events is grown under unstressed conditions as one control. Leaf samples at age of 3 w, 6 w, and 9 w, as well as along the period of drought treatment are collected for quantitative analysis of GUS activity. Inflorescence sample and mature seed from plants with and without drought treatment are also tested for GUS expression.

The results show, that the GUS expression is induced by drought stress compared to the control.

### Example 16

### Analysis of the Basal Activity of Promoter SEQ ID NO: 43

The basal activity of the promoter (SEQ ID NO: 43) in transgenic rice is tested by detecting the GUS expression level in callus, regenerated plantlets, and samples from the T0 plants growing in the greenhouse.

Twelve to twenty pieces of callus and about 12 regenerated plantlets transformed with any of the promoter with SEQ ID NO: 43 are sampled during rice transformation, and subjected for GUS staining analysis according to Jefferson et al., 1987.

Leaf samples of seedlings and adult plants, young inflorescence, and mature seed are collected from 20 to 25 single copy T0 plants per construct. Each sample is ground to powder at −80 degree Celsius and was then extracted with 50 mM phosphate buffer (pH7.0). The extracts are tested for their GUS activity via a kinetic fluorimetrical assay by using 4-methylumbelliferyl-beta-D-glucuronide (MUG) as substrate (Jefferson et al. 1987). The GUS activity in mature seeds is also examined by GUS staining. The results show no or hardly detectable basal GUS expression of the tested promoter (SEQ ID NO: 43) under unstressed conditions.

### Example 17

### Analysis of the Basal Activity of Promoter SEQ ID NO: 47

The basal activity of the promoter (SEQ ID NO: 47) in transgenic rice is tested by detecting the GUS expression level in callus, regenerated plantlets, and samples from the T0 plants growing in the greenhouse.

Twelve to twenty pieces of callus and about 12 regenerated plantlets transformed with any of the promoter with SEQ ID NO: 47 are sampled during rice transformation, and subjected for GUS staining analysis according to Jefferson et al., 1987.

Leaf samples of seedlings and adult plants, young inflorescence, and mature seed are collected from 20 to 25 single copy T0 plants per construct. Each sample is ground to powder at −80 degree Celsius and was then extracted with 50 mM phosphate buffer (pH7.0). The extracts are tested for their GUS activity via a kinetic fluorimetrical assay by using 4-methylumbelliferyl-beta-D-glucuronide (MUG) as substrate (Jefferson et al. 1987). The GUS activity in mature seeds is also examined by GUS staining. The results show no or hardly detectable basal GUS expression of the tested promoter (SEQ ID NO: 47) under unstressed conditions.

### Example 18

### Analysis of Promoter Activity in Response to Drought Treatment of Promoter SEQ ID NO: 43

The response of promoter with SEQ ID NO: 43 to drought treatment is examined in T1 transgenic rice. T1 seeds from 6 single-copy T0 events are checked for the expression of GFP, a visual marker present in the construct, and transgenic seeds are selected and sown in soil. Nine transgenic plants per event are grown in drought stressed regime. The same amount of plants from these events is grown under unstressed conditions as one control. Leaf samples at age of 3 w, 6 w, and 9 w, as well as along the period of drought treatment are collected for quantitative analysis of GUS activity. Inflorescence sample and mature seed from plants with and without drought treatment are also tested for GUS expression.

The results show, that the GUS expression is induced by drought stress compared to the control.

### Example 19

### Analysis of Promoter with Intron Activity in Response to Drought Treatment of

Promoter SEQ ID NO: 47 The response of promoter with SEQ ID NO: 47 to drought treatment is examined in T1 transgenic rice. T1 seeds from 6 single-copy T0 events are checked for the expression of GFP, a visual marker present in the construct, and transgenic seeds are selected and sown in soil. Nine transgenic plants per event are grown in drought stressed regime. The same amount of plants from these events is grown under unstressed conditions as one control. Leaf samples at age of 3 w, 6 w, and 9 w, as well as along the period of drought treatment are collected for quantitative analysis of GUS activity. Inflorescence sample and mature seed from plants with and without drought treatment are also tested for GUS expression.

The results show, that the GUS expression is induced by drought stress compared to the control.

TABLE 3
MATRIX and IUPAC string of the stress-induction element 1

| | Pos. | | | | | | | | | |
|---|---|---|---|---|---|---|---|---|---|---|
| | 1 | 2 | 3 | 4 | 5 | 6 | 7 | 8 | 9 | 10 |
| Matrix | | | | | | | | | | |
| A | 0.00 | 25.00 | 0.00 | 100.00 | 0.00 | 100.00 | 0.00 | 100.00 | 0.00 | 25.00 |
| C | 0.00 | 0.00 | 0.00 | 0.00 | 25.00 | 0.00 | 0.00 | 0.00 | 0.00 | 25.00 |
| G | 100.00 | 25.00 | 100.00 | 0.00 | 75.00 | 0.00 | 100.00 | 0.00 | 100.00 | 25.00 |
| T | 0.00 | 50.00 | 0.00 | 0.00 | 0.00 | 0.00 | 0.00 | 0.00 | 0.00 | 25.00 |
| IUPAC | G | D | G | A | G | A | G | A | G | N |

A = adenine;
C = cytosine;
G = guanine;
T = thymine;
U = uracil;
R = G A (purine);
Y = T C (pyrimidine);
K = G T (keto);
M = A C (amino);
S = G C;
W = A T;
B = G T C;
D = G A T;
H = A C T;
V = G C A;
N = A G C T (any)
The core region is defined by positions 3, 4, 6, 7, 8 and 9, preferably by positions 4, 6, 7, 8 and 9, most preferably by positions 6, 7, 8 and 9.

TABLE 4
MATRIX and IUPAC string of the stress-induction element 2

| | Pos. | | | | | | | | | | |
|---|---|---|---|---|---|---|---|---|---|---|---|
| | 1 | 2 | 3 | 4 | 5 | 6 | 7 | 8 | 9 | 10 | 11 |
| Matrix | | | | | | | | | | | |
| A | 5.60 | 27.80 | 0.00 | 100.00 | 0.00 | 0.00 | 27.80 | 0.00 | 38.90 | 16.70 | 24.20 |
| C | 33.30 | 33.30 | 100.00 | 0.00 | 100.00 | 0.00 | 5.50 | 100.00 | 5.50 | 22.70 | 27.40 |
| G | 50.00 | 16.70 | 0.00 | 0.00 | 0.00 | 100.00 | 0.00 | 0.00 | 55.60 | 21.70 | 24.20 |
| T | 11.10 | 22.20 | 0.00 | 0.00 | 0.00 | 0.00 | 66.70 | 0.00 | 0.00 | 38.90 | 24.20 |
| IUPAC | S | N | C | A | C | G | T | C | R | N | N |

A = adenine;
C = cytosine;
G = guanine;
T = thymine;
U = uracil;
R = G A (purine);
Y = T C (pyrimidine);
K = G T (keto);
M = A C (amino);
S = G C;
W = A T;
B = G T C;
D = G A T;
H = A C T;
V = G C A;
N = A G C T (any)
The core region is defined by positions 3, 4, 5, 6 and 8, preferably by positions 3, 4, 5 and 6.

TABLE 5
MATRIX and IUPAC string of the stress-induction element 3

| | Pos. | | | | | | | | |
|---|---|---|---|---|---|---|---|---|---|
| | 1 | 2 | 3 | 4 | 5 | 6 | 7 | 8 | 9 |
| Matrix | | | | | | | | | |
| A | 9.10 | 0.00 | 0.00 | 0.00 | 0.00 | 0.00 | 0.00 | 0.00 | 36.40 |
| C | 9.10 | 0.00 | 0.00 | 0.00 | 0.00 | 0.00 | 0.00 | 0.00 | 9.10 |
| G | 9.10 | 0.00 | 0.00 | 72.70 | 0.00 | 0.00 | 100.00 | 100.00 | 0.00 |
| T | 72.70 | 100.00 | 100.00 | 27.30 | 100.00 | 100.00 | 0.00 | 0.00 | 54.50 |
| IUPAC | T | T | T | G | T | T | G | G | W |
| | Pos. | | | | | | | | |
| | 10 | 11 | 12 | 13 | 14 | 15 | 16 | 17 | 18 |
| Matrix | | | | | | | | | |
| A | 0.00 | 18.20 | 9.10 | 72.70 | 18.20 | 27.30 | 9.10 | 9.10 | 45.40 |
| C | 36.40 | 36.40 | 36.40 | 0.00 | 36.40 | 0.00 | 27.30 | 27.30 | 36.40 |
| G | 27.20 | 27.20 | 9.10 | 9.10 | 0.00 | 18.20 | 9.10 | 0.00 | 0.00 |
| T | 36.40 | 18.20 | 45.40 | 18.20 | 45.40 | 54.50 | 54.50 | 63.60 | 18.20 |
| IUPAC | B | N | Y | A | Y | W | Y | T | M |

A = adenine;
C = cytosine;
G = guanine;
T = thymine;
U = uracil;
R = G A (purine);
Y = T C (pyrimidine);
K = G T (keto);
M = A C (amino);
S = G C;
W = A T;
B = G T C;
D = G A T;
H = A C T;
V = G C A;
N = A G C T (any)
The core region is defined by positions 2, 3, 5, 6, 7 and 8 preferably by positions 3, 5, 6, 7 and 8, most preferably by positions 5, 6, 7 and 8.

TABLE 6
MATRIX and IUPAC string of the stress-induction element 4

| | Pos. | | | | | | | | | | | | | | |
|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 |
| Matrix | | | | | | | | | | | | | | | |
| A | 15.80 | 15.80 | 5.30 | 31.60 | 15.80 | 26.30 | 0.00 | 0.00 | 0.00 | 0.00 | 57.90 | 5.30 | 0.00 | 26.30 | 15.80 |
| C | 36.80 | 63.20 | 47.40 | 26.30 | 63.20 | 26.30 | 94.70 | 0.00 | 100.00 | 100.00 | 0.00 | 94.70 | 100.00 | 26.30 | 52.60 |
| G | 5.30 | 0.00 | 10.50 | 0.00 | 5.20 | 15.80 | 0.00 | 0.00 | 0.00 | 0.00 | 5.30 | 0.00 | 0.00 | 5.30 | 10.60 |
| T | 42.10 | 21.00 | 36.80 | 42.10 | 15.80 | 31.60 | 5.30 | 100.00 | 0.00 | 0.00 | 36.80 | 0.00 | 0.00 | 42.10 | 21.00 |
| IUPAC | Y | C | Y | H | C | N | C | T | C | C | W | C | C | N | C |

A = adenine;
C = cytosine;
G = guanine;
T = thymine;
U = uracil;
R = G A (purine);
Y = T C (pyrimidine);
K = G T (keto);
M = A C (amino);
S = G C;
W = A T;
B = G T C;
D = G A T;
H = A C T;
V = G C A ;
N = A G C T (any)
The core region is defined by positions 7, 8, 9, 10 and 13, preferably by positions 7, 8, 9 and 10.

TABLE 7
MATRIX and IUPAC string of the stress-induction element 5

| | Pos. | | | | | | | | | | | | | | | | |
|---|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 | 17 |
| Matrix | | | | | | | | | | | | | | | | | |
| A | 75.00 | 75.00 | 25.00 | 25.00 | 50.00 | 50.00 | 0.00 | 50.00 | 0.00 | 75.00 | 12.50 | 75 | 100.00 | 0.00 | 100.00 | 100.00 | 25.00 |
| C | 12.50 | 12.50 | 25.00 | 37.50 | 37.50 | 12.50 | 25.00 | 12.50 | 12.50 | 0.00 | 50.00 | 25.00 | 0.00 | 100.00 | 0.00 | 0.00 | 37.50 |
| G | 0.00 | 0.00 | 37.50 | 25.00 | 0.00 | 12.50 | 75.00 | 12.50 | 75.00 | 12.50 | 37.50 | 0.00 | 0.00 | 0.00 | 0.00 | 0.00 | 25.00 |
| T | 12.50 | 12.50 | 12.50 | 12.50 | 12.50 | 25.00 | 0.00 | 25.00 | 12.50 | 12.50 | 0.00 | 0.00 | 0.00 | 0.00 | 0.00 | 0.00 | 12.50 |
| IUPAC | A | A | N | N | M | N | G | N | G | A | S | A | A | C | A | A | N |

A = adenine;
C = cytosine;
G = guanine;
T = thymine;
U = uracil;
R = G A (purine);
Y = T C (pyrimidine);
K = G T (keto);
M = A C (amino);
S = G C;
W = A T;
B = G T C;
D = G A T;
H = A C T;
V = G C A;
N = A G C T (any)
The core region is defined by positions 13, 14, 15 and 16.

TABLE 8
MATRIX and IUPAC string of the stress-induction element 6

| | Pos. | | | | | | | | | | | | | |
|---|---|---|---|---|---|---|---|---|---|---|---|---|---|---|
| | 1 | 2 | 3 | 4 | 5 | 6 | 7 | 8 | 9 | 10 | 11 | 12 | 13 | 14 |
| Matrix | | | | | | | | | | | | | | |
| A | 25.0 | 0.0 | 50.0 | 37.5 | 0.0 | 25.0 | 25.0 | 12.5 | 0.0 | 0.0 | 0.0 | 0.0 | 12.5 | 0.0 |
| C | 0.0 | 50.0 | 12.5 | 12.5 | 12.5 | 0.0 | 25.0 | 0.0 | 0.0 | 0.0 | 0.0 | 0.0 | 25.0 | 100.0 |
| G | 25.0 | 0.0 | 0.0 | 12.5 | 87.5 | 25.0 | 37.5 | 37.5 | 0.0 | 0.0 | 100.0 | 100.0 | 0.0 | 0.0 |
| T | 50.0 | 50.0 | 37.5 | 37.5 | 0.0 | 50.0 | 12.5 | 50.0 | 100.0 | 100.0 | 0.0 | 0.0 | 62.5 | 0.0 |
| IUPAC | D | Y | W | N | G | D | N | K | T | T | G | G | T | C |

| | Pos. | | | | | | | | | | | | |
|---|---|---|---|---|---|---|---|---|---|---|---|---|---|
| | 15 | 16 | 17 | 18 | 19 | 20 | 21 | 22 | 23 | 24 | 25 | 26 | 27 |
| Matrix | | | | | | | | | | | | | |
| A | 0.0 | 50.0 | 62.5 | 0.0 | 0.0 | 37.5 | 12.5 | 50 | 87.5 | 37.5 | 25.0 | 25.0 | 37.5 |
| C | 100.0 | 12.5 | 12.5 | 12.5 | 37.5 | 25.0 | 25.0 | 37.5 | 12.5 | 25.0 | 25.0 | 62.5 | 25.0 |
| G | 0.0 | 25.0 | 0.0 | 37.5 | 0.0 | 12.5 | 37.5 | 0.0 | 0.0 | 25.0 | 25.0 | 12.5 | 0.0 |
| T | 0.0 | 12.5 | 25.0 | 50.0 | 62.5 | 25.0 | 25.0 | 12.5 | 0.0 | 12.5 | 25.0 | 0.0 | 37.5 |
| IUPAC | C | N | A | K | Y | N | N | M | A | N | N | C | H |

A = adenine;
C = cytosine;
G = guanine;
T = thymine;
U = uracil;
R = G A (purine);
Y = T C (pyrimidine);
K = G T (keto);
M = A C (amino);
S = G C;
W = A T;
B = G T C;
D = G A T;
H = A C T;
V = G C A;
N = AGCT (any)
The core region is defined by positions 9, 10, 11, 12, 14 and 15, preferably by positions 9, 10, 11, 12 and 14, most preferably by positions 9, 10, 11 and 12.

TABLE 9
MATRIX and IUPAC string of the stress-induction element 7

| | Pos. | | | | | | | | | |
|---|---|---|---|---|---|---|---|---|---|---|
| | 1 | 2 | 3 | 4 | 5 | 6 | 7 | 8 | 9 | 10 |
| Matrix | | | | | | | | | | |
| A | 21.00 | 0.00 | 12.50 | 0.00 | 0.00 | 0.00 | 0.00 | 0.00 | 25.00 | 29.20 |
| C | 21.00 | 100.00 | 0.00 | 0.00 | 100.00 | 100.00 | 0.00 | 66.70 | 25.00 | 16.70 |
| G | 21.00 | 0.00 | 0.00 | 0.00 | 0.00 | 0.00 | 0.00 | 0.00 | 16.70 | 8.30 |
| T | 37.00 | 0.00 | 87.50 | 100.00 | 0.00 | 0.00 | 100.00 | 33.30 | 33.30 | 45.80 |
| IUPAC | N | C | T | T | C | C | T | Y | N | N |

A = adenine;
C = cytosine;
G = guanine;
T = thymine;
U = uracil;
R = G A (purine);
Y = T C (pyrimidine);
K = G T (keto);
M = A C (amino);
S = G C;
W = A T;
B = G T C;
D = G A T;
H = A C T;
V = G C A;
N = A G C T (any)
The core region is defined by positions 2, 4, 5, 6 and 7, preferably by positions 4, 5, 6 and 7.

TABLE 10
MATRIX and IUPAC string of the stress-induction element 8

| | Pos. | | | | | | | | | | | | | | | |
|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 |
| Matrix | | | | | | | | | | | | | | | | |
| A | 33.30 | 22.20 | 50.00 | 0.00 | 0.00 | 44.40 | 0.00 | 0.00 | 100.00 | 0.00 | 33.30 | 33.30 | 27.80 | 27.80 | 33.30 | 38.90 |
| C | 16.70 | 11.10 | 0.00 | 0.00 | 0.00 | 0.00 | 0.00 | 0.00 | 0.00 | 0.00 | 11.10 | 5.60 | 0.00 | 11.10 | 0.00 | 5.60 |
| G | 38.90 | 50.00 | 22.20 | 100.00 | 94.40 | 0.00 | 100.00 | 100.00 | 0.00 | 100.00 | 27.80 | 50.00 | 44.40 | 55.60 | 50.00 | 38.90 |
| T | 11.10 | 16.70 | 27.80 | 0.00 | 5.60 | 55.60 | 0.00 | 0.00 | 0.00 | 0.00 | 27.80 | 11.10 | 27.80 | 5.50 | 16.70 | 16.60 |
| IUPAC | N | N | W | G | G | W | G | G | A | G | N | R | D | R | R | R |

A = adenine;
C = cytosine;
G = guanine;
T = thymine;
U = uracil;
R = G A (purine);
Y = T C (pyrimidine);
K = G T (keto);
M = A C (amino);
S = G C;
W = A T;
B = G T C;
D = G A T;
H = A C T;
V = G C A;
N = A G C T (any)
The core region is defined by positions 4, 5, 7, 8, 9 and 10, preferably by positions 5, 7, 8, 9 and 10, most preferably by positions 7, 8, 9 and 10.

TABLE 11
Preferred combinations of SIEs in the promoters of the invention

| | | | | | | | | | examplary |
|---|---|---|---|---|---|---|---|---|---|
| Stress Inducible Element | 1 | 2 | 3 | 4 | 5 | 6 | 7 | 8 | sequence |
| preferred Combination 1 | | | 4 times | 4 times | | | | | Seq ID No |
| | | | | | | | | | 41 and 45 |
| preferred Combination 2 | | | | 4 times | | | 4 times | | Seq ID No |
| | | | | | | | | | 42 and 46 |
| preferred Combination 3 | | 4 times | | | 4 times | 4 times | | | Seq ID No |
| | | | | | | | | | 43 and 47 |
| preferred Combination 4 | 4 times | | 4 times | | | | | | |
| preferred Combination 5 | | | 4 times | | | | | 4 times | |
| preferred Combination 6 | | 4 times | | | | 4 times | | | |
| preferred Combination 7 | | 4 times | 4 times | | | | | | |
| preferred Combination 8 | | | | | 4 times | 4 times | | | |
| preferred Combination 9 | | | | 4 times | | | 4 times | | |
| preferred Combination 10 | 4 times | | | | | | 4 times | | |
| preferred Combination 11 | | | | | | | 4 times | 4 times | |
| preferred Combination 12 | 4 times | 4 times | | | | | | | |
| preferred Combination 13 | | 4 times | | 4 times | | | | | |
| preferred Combination 14 | | 4 times | | | | | | 4 times | |
| preferred Combination 15 | | | 4 times | | | 4 times | | 4 times | |
| preferred Combination 16 | 4 times | | 4 times | | | 4 times | | | |
| preferred Combination 17 | | | 4 times | 4 times | | 4 times | | | |
| preferred Combination 18 | | | 4 times | | 4 times | 4 times | | | |

1. A recombinant promoter molecule for regulating stress-induced expression comprising at least one stress-induction element (SIE) or the complement or reverse complement thereof, wherein the SIE comprises a sequence as defined by the matrices in tables 3 to 10, wherein the matrix similarity is at least 0.8 and the core similarity is at least 0.75, wherein said recombinant promoter molecule is functionally linked to a minimal promoter heterologous to the at least one SIE.

2. The recombinant promoter molecule for regulating stress-inducible expression of claim 1 wherein the at least one SIE has a nucleic acid sequence selected from SEQ ID NOS: 1, 2, 3, 6, 7, 8, 11, 12, 13, 16, 17, 18, 21, 22, 23, 26, 27, 28, 31, 32, 33, 36, 37 and 38, or the complement or reverse complement thereof.

3. The recombinant promoter molecule of claim 1, wherein the recombinant promoter molecule comprising said at least one SIE regulates stress-induced expression of heterologous nucleic acid molecules to which the recombinant promoter molecule is functionally linked.

4. The recombinant promoter molecule of claim 1, wherein said recombinant promoter molecule is selected from the group consisting of:
a) a nucleic acid molecules having a nucleic acid sequence selected from SEQ ID NOS: 4, 9, 14, 19, 24, 29, 34, 39, 41, 42, 43, 45, 46, 47, 48, 49, 50, 51, 52, 53, 54 and 55;
b) a nucleic acid molecule having a nucleic acid sequence with an identity of at least 60% to a sequence selected from SEQ ID NOS: 4, 9, 14, 19, 24, 29, 34, 39, 41, 42, 43, 45, 46, 47, 48, 49, 50, 51, 52, 53, 54 and 55;
c) a fragment of 150 or more consecutive bases of a nucleic acid molecule of a) or b); or
d) a nucleic acid molecule of 150 nucleotides or more, hybridizing under conditions equivalent to hybridization in 7% sodium dodecyl sulfate (SDS), 0.5 M NaPO₄, 1 mM EDTA at 50° C. with washing in 2×SSC, 0.1% SDS at 50° C. to a nucleic acid molecule comprising at least 100 consecutive nucleotides of a promoter having a nucleic acid sequence selected from SEQ ID NOS: 4, 9, 14, 19, 24, 29, 34, 39, 41, 42, 43, 45, 46, 47, 48, 49, 50, 51, 52, 53, 54 and 55 or a complement thereof; or
e) a nucleic acid molecule which is the complement or reverse complement of any of the previously mentioned nucleic acid molecules under a) to d); or
wherein the promoter molecules as defined under b) to e) cause stress-induced expression of heterologous nucleic acid molecules to which they are functionally linked.

5. A recombinant expression construct comprising one or more recombinant promoter molecules of claim 1.

6. The recombinant expression construct of claim 5, wherein the promoter molecule is functionally linked to one or more nucleic acid molecule to be expressed, the latter being heterologous to the recombinant promoter molecule to which it is functionally linked.

7. A recombinant expression vector comprising one or more recombinant expression constructs of claim 5.

8. A transgenic cell, a transgenic plant or part thereof comprising one or more recombinant promoter molecules of claim 1.

9. The transgenic cell, transgenic plant or part thereof of claim 8, selected or derived from the group consisting of bacteria, fungi, animals and plants.

10. A transgenic cell culture, transgenic seed, parts or propagation material derived from the transgenic cell or plant or part thereof of claim 8.

11. A method for the identification and isolation of promoter molecules for stress-induced expression comprising the steps of:
A) identifying a promoter molecule in genomic DNA;
B) identifying promoters of A) that contain any of the matrices as defined in tables 3 to 10 or any of the SIEs as defined by SEQ ID NOS: 1, 2, 3, 6, 7, 8, 11, 12, 13, 16, 17, 18, 21, 22, 23, 26, 27, 28, 31, 32, 33, 36, 37 or 38 using any computational SIE detection or IUPAC string matching sequence analysis tools; and
C) isolating at least 250 bp of genomic DNA comprising said SIEs and a minimal promoter.

12. A method for providing stress-inducible promoter molecules comprising the step of functionally linking at least one SIE having a sequence selected from SEQ ID NOS: 1, 2, 3, 6, 7, 8, 11, 12, 13, 16, 17, 18, 21, 22, 23, 26, 27, 28, 31, 32, 33, 36, 37 and 38 to a minimal promoter.

13. The method of claim 12, wherein the minimal promoter is comprised in a bacterial, viral, animal or plant promoter.

14. The method of claim 13 wherein the promoter is a plant promoter.

15. (canceled)
